(19) United States
(12) Patent Application Publication
(10) Pub. No.: US 2024/0366858 A1
(43) Pub. Date: Nov. 7, 2024

(54) INTRAVENOUS INFUSION PUMP WITH DISPLAY SCREEN HAVING MULTIPLE MACHINE-READABLE CODES FOR MULTIPLE PUMP DRIVERS

(71) Applicant: ICU Medical, Inc., San Clemente, CA (US)

(72) Inventors: Robert P. Cousineau, Boston, MA (US); Kerin L. Klagges-Kingsbury, Boxford, MA (US); James L. Cudney, Santee, CA (US); Roger P. Soucy, III, Kingston, NH (US)

(21) Appl. No.: 18/776,838

(22) Filed: Jul. 18, 2024

Related U.S. Application Data
(63) Continuation of application No. 17/932,241, filed on Sep. 14, 2022.
(60) Provisional application No. 63/254,922, filed on Oct. 12, 2021.

(51) Int. Cl. A61M 5/14; A61M 5/142; A61M 5/172

(52) U.S. Cl. CPC A61M 5/1413; A61M 5/1422; A61M 5/172; A61M 2205/12; A61M 2205/502; A61M 2205/8206

(57) ABSTRACT

Disclosed in some embodiments is an electronic intravenous infusion pump provided with a disposable, insertable pump cartridge that is connectable to one or more intravenous fluid infusion sources. The infusion pump has multiple pump drivers and an electronic display with multiple display regions. Each display region includes a graphic associating information in that region with a different one of the pump drivers. Each display region also includes a machine-readable code.

### RELATED APPLICATIONS

This is a continuation of U.S. patent application Ser. No. 17/932,241, filed on Sep. 14, 2022 and entitled “INTRAVENOUS INFUSION PUMP WITH CASSETTE INSERTION AND PUMP CONTROL USER INTERFACE,” which claims the benefit under 35 U.S.C. § 119 (e) of U.S. Provisional Patent Application No. 63/254,922, filed on Oct. 12, 2021 and entitled “INTRAVENOUS INFUSION PUMP WITH CASSETTE INSERTION AND PUMP CONTROL USER INTERFACE,” the entire contents of all of which are hereby incorporated by reference herein and made a part of this specification for all that they disclose.

### BACKGROUND

### Field

This disclosure relates to intravenous infusion pumps, including electronically controlled intravenous infusion pumps.

### Related Art

Patients all over the world who are in need of medical care commonly receive intravenous infusion therapy, especially during surgery or when hospitalized. This process generally involves inserting a needle into a patient's blood vessel, usually in the hand or arm, and then coupling the needle to a catheter in communication with one or more different types of therapeutic fluids. Once connected, the fluid travels from the fluid source(s), through the catheter, and into the patient. The fluid can provide certain desired benefits to the patient, such as maintaining hydration or nourishment, diminishing infection, reducing pain, lowing the risk of blood clots, maintaining blood pressure, providing chemotherapy, and/or delivering any other suitable drug or other therapeutic liquid to the patient. Electronic infusion pumps in communication with the fluid sources and the patient can help to increase the accuracy and consistency of fluid delivery to patients, but current electronic infusion pumps have disadvantages.

### SUMMARY

In some embodiments, an electronic intravenous infusion pump is provided with a disposable, insertable pump cartridge that is connected to one or more intravenous fluid infusion sources, wherein a user interface on a user communicator of the pump (such as a display/input device) interacts with and responds to the user's insertion of the cassette.

In some implementations, a medical infusion pump system can include an electronic processor with an electronic memory; an electrical power cable or battery; an electromechanical pump driver configured to receive a disposable fluid holder and to pump medical fluid through the fluid holder; and an electronic display. The pump driver can generate a signal indicating whether the fluid holder has been received by the pump driver. The electronic processor can be configured to retrieve from the electronic memory and show on the electronic display one or more repeating moving graphics or animations with a representation of the fluid holder being inserted into the pump, until the processor confirms that the fluid holder has been received by the pump in response to the signal generated by the pump driver.

In some implementations, A medical infusion pump system can include an electronic processor with an electronic memory; an electrical power cable or battery; an electromechanical pump driver configured to receive at least one disposable fluid holder and to pump medical fluid through the fluid holder, the at least one fluid holder being connectable to one or more fluid lines from one or more fluid source containers; and an electronic display comprising a sensing region configured to detect a user's touch selection. The electronic processor can be configured to retrieve from the electronic memory and show on the electronic display a graphic that includes a representation of the least one fluid holder and a correlation between the at least one fluid holder and the sensing region.

In some implementations, a medical infusion pump system can include an electronic processor with an electronic memory; an electrical power cable or battery; an electromechanical pump driver configured to receive at least one disposable fluid holder and to pump medical fluid through the fluid holder; and an electronic display comprising a sensing region configured to detect a user's touch selection. The fluid holder can be connectable to one or more fluid lines from one or more fluid source containers. The display can be configured to permit a user to input multiple pumping stages comprising one or more different pumping parameters to be performed sequentially automatically by the pump. The display can be configured to show multiple representations of the pumping stages simultaneously on the display.

### BRIEF DESCRIPTION OF THE DRAWINGS

The following drawings and the associated descriptions are provided to illustrate embodiments of the present disclosure and do not limit the scope of the claims.

FIGS. 1A-E show front perspective, front elevational, rear elevational, top plan, and side elevational views, respectively, of an example of an infusion pump.

FIG. 2A shows an example of a cassette that can be used with the pump of FIG. 1.

FIGS. 2B-2D shows an example of a cassette that is the same as or similar to the cassette of FIG. 2A that can be used with the pump of FIG. 1.

FIG. 3A illustrates components of a pump driver that can interact with the cassette(s) of FIGS. 2A-2D.

FIG. 3B illustrates a fluid path through a cassette such as one or more of those shown in FIGS. 2A-2D, such as may be controlled by the hardware of FIG. 3A.

FIG. 3C illustrates schematically how hardware (e.g., FIG. 3A) interacts with a cassette (e.g., FIGS. 2A-2D) to affect flow along a fluid path.

FIG. 3D shows an example of a schematic diagram of some functional components of a medical pump system that can be used with or instead of those illustrated or described elsewhere in this application.

FIGS. 4A-4F show an example of a graphical user interface of a user communicator, such as display/input device, urging a user to insert a cassette into the pump driver.

FIG. 5 shows an example of another graphical user interface of a user communicator, permitting a user to select options for programming medical fluid infusion.

FIGS. 6A-8B show example of other graphical user interfaces of a user communicator, permitting a user to enter and/or to select parameters for medical fluid infusion.

FIG. 9 shows an example of a graphic user interface of a user communicator during a pumping phase.

### DETAILED DESCRIPTION

This specification provides textual descriptions and illustrations of many devices, components, assemblies, and subassemblies. Any structure, material, function, method, or step that is described and/or illustrated in one example can be used by itself or with or instead of any structure, material, function, method, or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification, but should not be considered to limit any claim unless recited in such claim.

### Examples of Pump Systems

In some embodiments, a pump system can include a reusable pump driver and a disposable fluid holder, such as a fluid cassette, syringe, section of tubing, etc. A disposable cassette, which is typically adapted to be used only once for a single patient and/or only once for one fluid delivery cycle, is usually a small plastic unit having at least one inlet and an outlet respectively connected through flexible tubing to the fluid supply container and intravenously through a needle to the patient receiving the fluid. In some embodiments, the cassette can include a pumping chamber. The flow of fluid through the chamber can be controlled by a plunger or pumping element activated in a controlled manner by the pump driver. For example, the cassette chamber can have one wall formed by a flexible diaphragm against which the plunger is repeatedly pressed in a reciprocating manner, which causes the fluid to flow. The pump driver can include the plunger or pumping element for controlling the flow of fluid into and out of the pumping chamber in the cassette, and it may also include one or more controls and/or vents to help deliver the fluid to the patient at a pre-set rate, in a pre-determined manner, for a particular pre-selected time, and/or at a pre-selected total dosage.

In some embodiments, the fluid can enter a cassette through an inlet and can be forced through an outlet under pressure. The fluid is delivered to the outlet when the pump plunger forces the membrane into the pumping chamber to displace the fluid. During the intake stroke, the pump plunger draws back, the membrane covering the pumping chamber retracts or pulls back from its prior inwardly displaced position, and the fluid is then drawn through the open inlet and into the pumping chamber. In a pumping stroke, the pump plunger forces the membrane back into the pumping chamber to force the fluid contained therein through the outlet. By repeating this action in an electronically controlled manner, the fluid flows into and out of the cassette in a series of spaced-apart pulses rather than in a continuous flow. When the pulses occur in rapid succession, the flow approximates a continuous flow. The entire disclosure of U.S. Pat. No. 7,258,534 is incorporated by reference herein, for all purposes, for all that it contains, including but not limited to examples of pump drivers and disposable fluid holders. It is contemplated that any structure, material, function, method, or step that is described and/or illustrated in the '534 patent can be used with or instead of any structure, material, function, method, or step that is described and/or illustrated in the text or drawings of this specification.

### Examples of Pump System Components

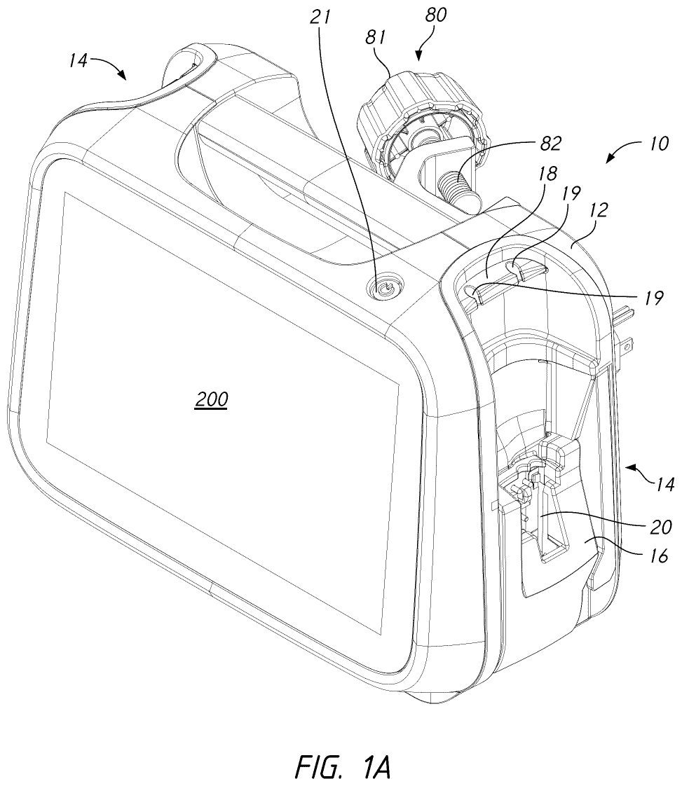

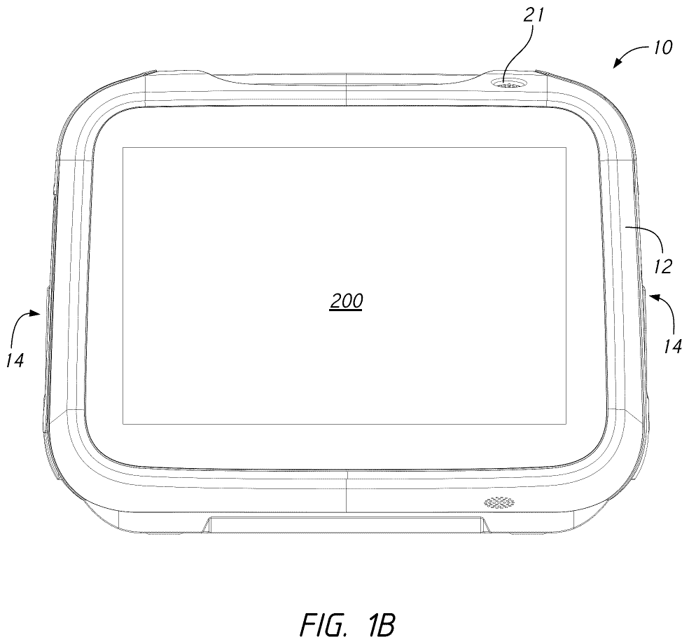

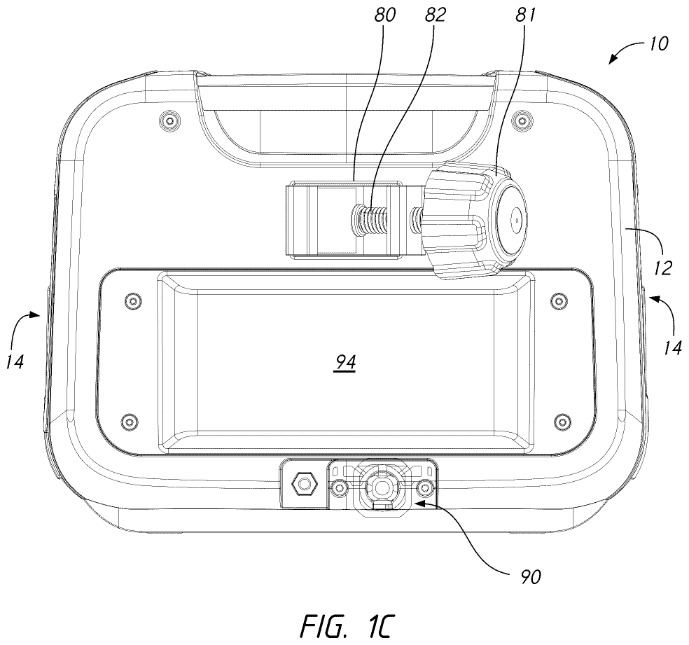

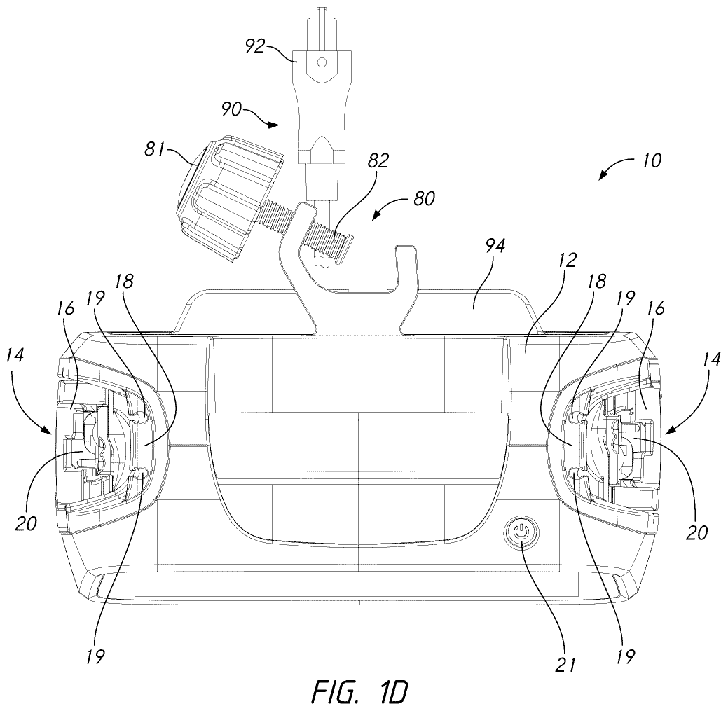

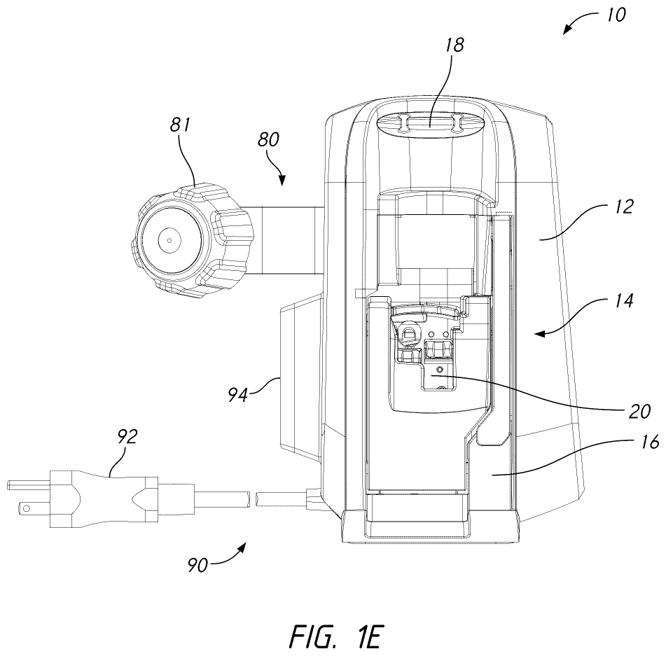

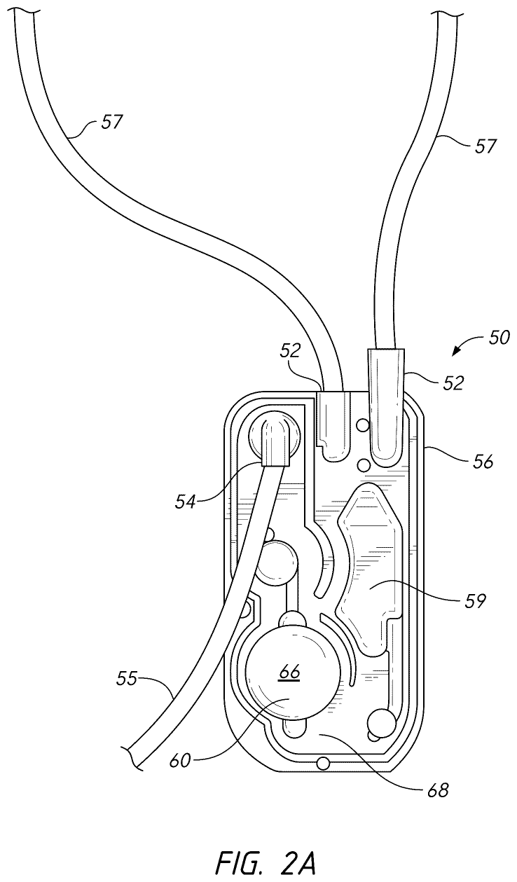

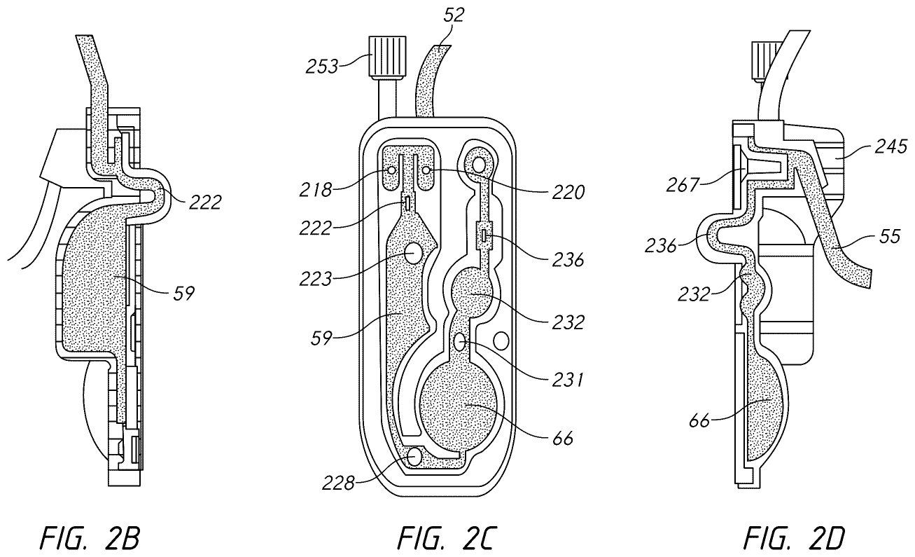

FIGS. 1A-1E show an electronic medical intravenous pump 10 with a housing 12 and at least one electromechanical pump driver 14 attached to the housing 12. As illustrated, a plurality of pump drivers 14 (e.g., at least two) can be integrally provided within the same housing 12 of a single medical pump 10. Either or both of the pump drivers 14 can include a cover 16 that partially or entirely encloses an outer surface of the pump driver 14, an indicator 18 (e.g., an illuminating communicator) attached to the cover 16, one or more tube holders 19, and a loader 20 configured to securely receive and releasably hold a disposable fluid holder (see, e.g., FIGS. 2A-2D), including but not limited to a cassette, syringe, and/or tubing. The one or more tube holders 19 can be configured to removably receive and securely hold one or more fluid-conveying tubes extending into or exiting from fluid holder when the fluid holder is received into the loader 20. The indicator 18 can communicate one or more messages to a user, such as by temporarily illuminating in one or more colors. Examples of one or more message include confirming that a pump driver 14 near the indicator is currently active and pumping or that one or more instructions being received from a user will apply to a pump driver 14 near the indicator 18. The loader 20 can be a mechanism with multiple moving parts that opens, closes, expands, contracts, clasps, grasps, releases, and/or couples with the fluid holder to securely hold the fluid holder on or within the pump 10 during fluid pumping into the patient. The loader 20 can be integrated into and positioned on or within the pump 10 near the cover 16 adjacent to the indicator 18.

A user communicator, such as display/input device 200, can be provided to convey information to and/or receive information from a user (e.g., in an interactive manner). As illustrated, the user communicator is a touch screen that is configured to provide information to a user through an illuminated dynamic display and is configured to sense a user's touch to make selections and/or to allow the user to input instructions or data. For example, the display-input device 200 can permit the user to input and see confirmation of the infusion rate, the volume of fluid to be infused (VTBI), the type of drug being infused, the name of the patient, and/or any other useful information. The display-input device 200 can be configured to display one or more pumping parameters on a continuing basis, such as the name of the drug being infused, the infusion rate, the volume that has been infused and/or the volume remaining to be infused, and/or the elapsed time of infusion and/or the time remaining for the programmed course of infusion, etc. As shown, the touch screen can be very large, for example at least about 4 inches×at least about 6 inches, or at least about 6 inches×at least about 8 inches. In the illustrated example, the touch screen fills substantially the entire front surface of the pump 10 (see FIG. 1A), with only a small protective boundary surrounding the touch screen on the front surface. As shown, the touch screen comprises at least about 80% or at least about 90% of the surface area of the front of the pump 10. In some implementations, the front of the touch screen comprises a clear glass or plastic plate that can be attached to the housing 20 in a manner that resists liquid ingress, such as using a water-proof gasket and/or adhesive that can withstand repeated exposure to cleaning and sanitizing agents commonly used in hospitals without significant degradation.

An actuator 21 can be provided separate from the user communicator. The actuator 21 can be configured to receive an input and/or display information to a user. As shown, the actuator 21 is a power button that permits the user to press on the actuator 21 to power up the pump 10. The actuator 21 can illuminated to communicate to the user that the pump 10 is power on. If the power source is running low, the actuator 21 can change the color of illumination to quickly show to a user that a power source needs to be replenished.

In some embodiments, the user communicator, such as a display/input device 200, can alternatively or additionally comprise one or more screens, speakers, lights, haptic vibrators, electronic numerical and/or alphabetic read-outs, keyboards, physical or virtual buttons, capacitive touch sensors, microphones, and/or cameras, etc.

During use, the pump 10 is typically positioned near the patient who is receiving fluid infusion from the pump 10, usually lying in a bed or sitting in a chair. In some embodiments, the pump 10 may be configured to be an ambulatory pump, which will typically include a smaller housing, user communicator, battery, etc., so as to be conveniently transportable on or near a mobile patient. In many implementations, the pump 10 is attached to an IV pole stand (not shown) adjacent to the patient's bed or chair. As shown, the pump 10 can include a connector 80 that is configured to removably attach the pump 10 to the IV pole stand. As shown, the connector 80 can comprise an adjustable clamp with a large, casily graspable user actuator, such as a rotatable knob 81, that can be configured to selectively advance or retract a threaded shaft 82. At an end of the shaft 82 opposite from the knob 81 is a pole-contacting surface that can be rotatably advanced by the user to exert a force against a selected region of the pole, tightly pushing the pole against a rear surface of the pump 10, thereby securely holding the pump 10 in place on the pole during use. The selected region of the pole where the contacting surface of the shaft 82 is coupled can be chosen so as to position the pump 10 at a desired height for convenient and effective pumping and interaction with the patient and user.

The pump 10 can include a power source 90. In some embodiments, the power source can comprise one or more channels for selectively supplying power to the pump 10. For example, as illustrated, the power source 90 can comprise an electrical cable 92 configured to be attached to an electrical outlet and/or a portable, rechargeable battery 94. One or more components of the pump 10 can operate using either or both sources of electrical power. The electrical cable 92 can be configured to supply electrical power to the pump 10 and/or supply electrical power to the battery 94 to recharge or to maintain electrical power in the battery 94.

Inside of the housing 20 of the pump 10, various electrical systems can be provided to control and regulate the pumping of medical fluid by the pump 10 into the patient and/or to communicate with the user and/or one or more other entities. For example, the pump 10 can include a circuit board that includes a user interface controller (UIC) configured to control and interact with a user interface, such as a graphical user interface, that can be displayed on the user communicator or display/input device 200. The pump 10 can include a printed circuit board that includes a pump motor controller (PMC) that controls one or more pump drivers 14. In some embodiments, the PMC is located on a separate circuit board from the UIC and/or the PMC is independent from and separately operable from the UIC, each of the PMC and UIC including different electronic processors capable of concurrent and independent operation. In some embodiments, there are at least two PMC's provided, a separate and independent one for each pump driver 14, capable of concurrent and independent operation from each other. The pump 10 can include a printed circuit board that includes a communications engine (CE) that controls electronic communications between the pump 10 and other entities (aside from the user), such as electronic, wired or wireless, communication with a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The CE can include or can be in electronic communication with an electronic transmitter, receiver, and/or transceiver capable of transmitting and/or receiving electronic information by wire or wirelessly (e.g., by Wi-Fi, Bluetooth, cellular signal, etc.). In some embodiments, the CE is located on a separate circuit board from either or both of the UIC and/or the PMC(s), and/or the CE is independent from and separately operable from either or both of the UIC and/or the PMC(s), each of the PMC(s), UIC, and CE including different electronic processors capable of concurrent and independent operation. In some embodiments, any, some, or all of the UIC, CE, and PMC(s) are capable of operational isolation from any, some, or all of the others such that it or they can turn off, stop working, encounter an error or enter a failure mode, and/or reset, without operationally affecting and/or without detrimentally affecting the operation of any, some, or all of the others. In such an operationally isolated configuration, any, some, or all of the UIC, CE, and PMC(s) can still be in periodic or continuous data transfer or communication with any, some, or all of the others. The UIC, PMC(s), and/or CE can be configured within the housing 20 of the pump 10 to be in electronic communication with each other, transmitting data and/or instructions between or among each of them as needed.

FIG. 2A shows an example of a disposable fluid holder, such as a disposable cassette 50, that includes a plastic housing and a flexible, elastomeric silicon membrane. Any structure, material, function, method, or step that is described and/or illustrated in U.S. Pat. No. 4,842,584, which is incorporated herein by reference in its entirety, including but not limited to the pumping cassette, can be used by itself or with or instead of any structure, material, function, method, or step that is described and/or illustrated in this specification. The plastic housing of the cassette 50 can include one or more (e.g., two as shown) fluid inlets 52 and a fluid outlet 54 formed in a main body 56. The cassette 50 can be temporarily positioned for example in the loader 20 of a pump driver 14. The one or more fluid inlets 52 are coupled with one or more inlet tubes 57 in fluid communication with one or more sources of medical fluid, such as one or more IV bags, vials, and/or syringes, etc., containing medical fluid. If multiple inlets 52 and inlet tubes 57 are provided, as shown, then multiple sources of medical fluid can be simultaneously supplied to a patient through the cassette 50. The fluid outlet 54 is coupled to an outlet tube 55 in fluid communication with the patient, normally by way of a needle leading into a patient's blood vessel.

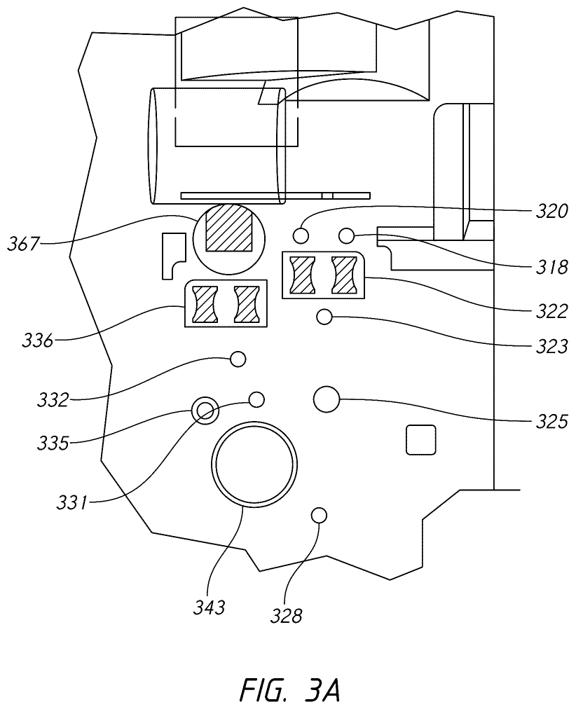

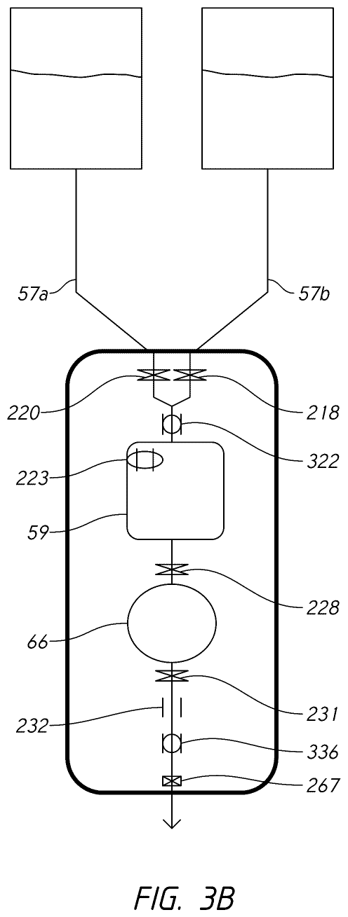

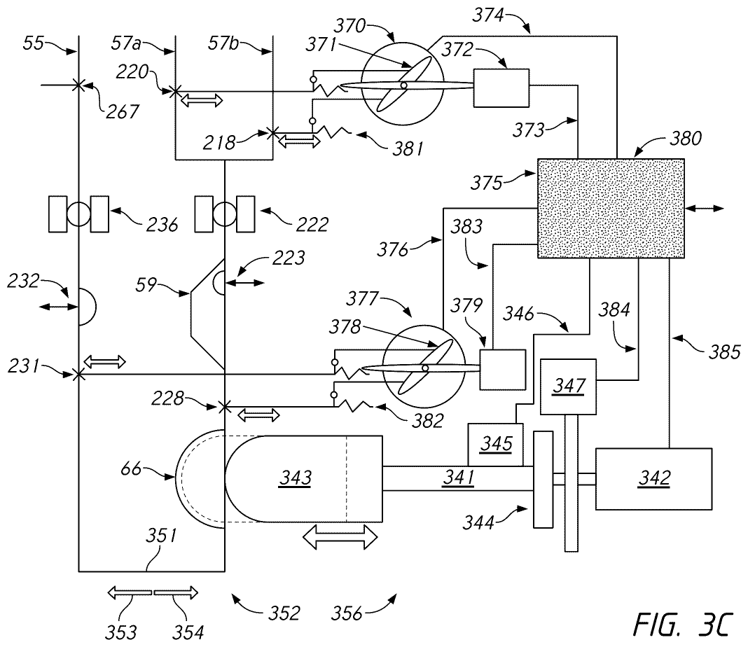

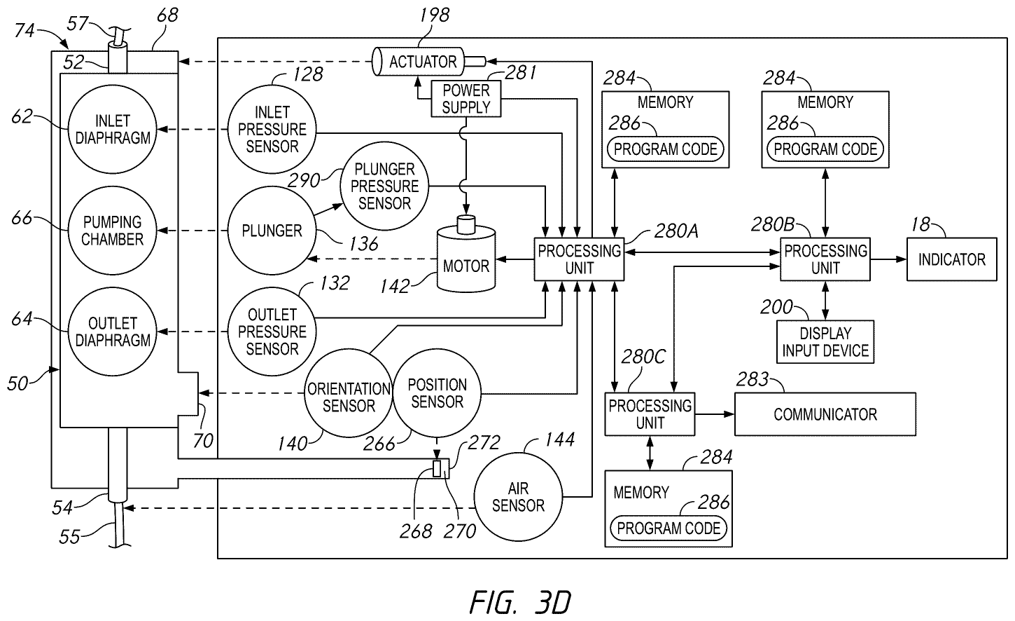

A flexible, elastomeric membrane forms a diaphragm 60 within a pumping chamber 66 on an inner face 68 of the main body 56. In operation, fluid enters through one or more of the inlets 52 and is forced through the outlet 54 under pressure. One or more fluid channels within the main body 56 of the cassette 50 convey the fluid between the inlets 52 and the outlet 54 by way of the pumping chamber 66. Before use, the cassette is typically primed with fluid, usually saline solution. A volume of fluid is delivered to the outlet 54 when a plunger 136 of the pump 10 (see, e.g., FIG. 3) displaces the diaphragm to expel the fluid from the pumping chamber 66. During an intake stroke, the plunger 136 retracts from the diaphragm 60, and the fluid is then drawn in through the inlet 52 and into the pumping chamber 66. In a pumping stroke, the pump 10 displaces the diaphragm 60 of the pumping chamber 66 to force the fluid contained therein through the outlet 54. In some embodiments, the directional movement of flow can be facilitated by one or more directional valve(s) (e.g., at one or more of inlet 52 or outlet 54). The fluid can flow from the cassette 50 in a series of spaced-apart pulses rather than in a continuous flow. In some embodiments, the pump 10 can deliver fluid to a recipient (e.g., a patient) at a pre-set rate, in a pre-determined manner, and for a particular (e.g., pre-selected) time or total dosage. The cassette 50 can include an air trap 59 in communication with an air vent (not shown).

FIGS. 2B, 2C, and 2D show three views of a cassette that is the same as or similar to the cassette of FIG. 2A. In FIGS. 2B and 2C, fluid can flow into an inlet 52, from a primary container, for example. Fluid can also flow into a secondary port 253, which can have a Y-connector with a resealable opening or a locking cap. Fluid coming in from the inlet 52 can pass through an A valve 220. Fluid coming in through a secondary port 253 can pass through a B valve 218. Fluid coming in through these two valves can then pass by a proximal air-in-line sensor 222. Fluid can then pass by, in a widening passage, a proximal pressure sensor 223.

### Cassette Air Trap

The widened passage can form an air trap chamber 59, which can allow for fluid mixing. The air trap chamber is also shown in the side view of FIG. 2B. The air trap chamber 59 can be integral to the cassette. The air trap can be exposed to view above the upper edge of the cassette door when the door is closed. Air passes the proximal air-in-line sensor 222 before entering the air trap, which in some embodiments can have a volume of at least about 2.0 mL (e.g., 2.15 mL). The proximal pressure sensor (see, e.g., pressure sensor 223 of FIG. 3C) can monitor pressure in the air trap chamber 59. In some embodiments, the user can remove air or fluid from the proximal tubing and cassette air trap after the cassette door is closed. To remove air in the trap or the proximal tubing the user may be required to attach a container to a Line B port (e.g., secondary port 253 of FIG. 2C). A key, button, or other control (e.g., on an infuser display screen) can be selected to backprime when a delivery is not in progress. When the user selects backprime, for example, this can initiate rapid pumping of fluid from Line A to a user-attached container on Line B. In some embodiments, no fluid is delivered to the cassette distal line during a backprime. After the backprime control is released, a cassette leak test can be automatically performed.

In some embodiments, after passing through an air trap chamber 59, fluid can subsequently flow through an inlet valve 228 and from there into a pumping chamber 66. The pumping chamber 66 is also shown in the side view of FIG. 2D. From the pumping chamber 66, fluid can flow through an outlet valve 231 and then into a widened passage accessed by a distal pressure sensor 232. This passage subsequently narrows down to pass a distal air-in-line sensor 236. The two air-in-line sensors, proximal 222, and distal 236, can both be positioned near a bend in a passage or tubing, as shown in the side views of FIGS. 2B and 2D. Fluid can flow through or pass a precision gravity flow regulator 267, seen in FIG. 2D. A finger grip 245 is also seen protruding to the right in FIG. 2D. An outlet tube 55 is also shown coming from the precision gravity flow regulator 267 and leading to a patient. The features shown in the cross sectional schematics of FIGS. 2B-2D can correspond generally to the external cassette contours shown in FIG. 2A.

### Fluid Delivery

A pumping system or infuser can deliver fluids from one or two drug sources through a sterile fluid pathway of administration set tubing, accessories and a cassette. In some embodiments, there is no contact between the fluid and an infusion mechanism subsystem (see FIG. 3A and the electromechanical portion 356 of FIG. 3C).

A system user can enter a multi-step therapy program to perform an infusion in a sequence of different delivery rates and volumes. The user can also enter a piggyback therapy program that sequentially delivers fluid from Line B and Line A. Line B starts delivering first and after Line B completes delivery, then Line A delivery is automatically started.

Alternatively, fluid from lines A and B can be interspersed or delivered simultaneously but at different rates such that a consistent ratio is maintained between the substances. For example, a concurrent therapy program can combine fluid from both Line A and Line B in the cassette pumping chamber during each chamber fill cycle, then deliver a combination of the two fluids with each plunger stroke.

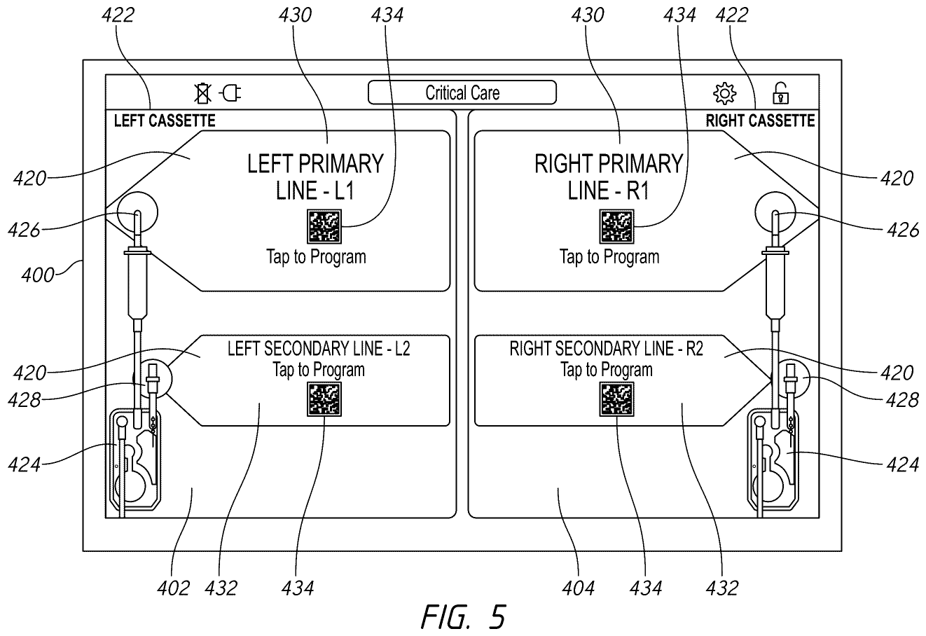

An additional or alternative infusion pump cassette that can be used with any embodiment in this specification is illustrated in FIG. 5 of U.S. Pat. No. 7,402,154. An elastomeric membrane 60 forms an inlet diaphragm 62, an outlet diaphragm generally indicated at 64, and a pumping chamber 66 located between the inlet and outlet diaphragms 62 and 64 on an inner face 68 of the main body 56. In operation, fluid enters through the inlet 52 and is forced through outlet 54 under pressure. The fluid is delivered to the outlet 54 when the plunger 136 of the pump 10 displaces the pumping chamber 66 to expel the fluid. During the intake stroke the plunger 136 releases the pumping chamber 66, and the fluid is then drawn through the inlet 52 and into the pumping chamber 66. In a pumping stroke, the pump 10 displaces the pumping chamber 66 to force the fluid contained therein through the outlet 54. The directional movement of flow can be facilitated by one or more directional valve(s) (e.g., at one or more of inlet 52 or outlet 54). At low rates the flow can be delivered in discrete volumes as the pump 10 displaces the pump chamber in successive steps. Thus, the fluid can flow from the cassette 50 in a series of spaced-apart pulses rather than in a smoothly continuous flow. Typically, this pump can deliver fluid to a recipient (e.g., a patient) at a pre-set rate, in a pre-determined manner, and for a particular (e.g., pre-selected) time or total dosage. A flow stop can be formed as a switch in a main body and protrude from the inner surface 68. This protrusion can form an irregular portion of the inner surface 68 which can be used to align the cassette 50 as well as monitor the orientation of the cassette 50. The flow stop can provide a manual switch for closing and opening the cassette 50 to fluid flow. A rim 72 is located around the outer surface of the main body 56 and adjacent the inner surface 68. The rim 72 can be used to secure the cassette in a fixed position relative to the pump 10 of U.S. Pat. No. 7,402,154.

FIG. 3A illustrates an example of hardware or components of the pump driver 14 that can be configured to interact with a fluid holder such as the cassette of FIGS. 2A-2D. In FIG. 3A, an A valve interface 320 can correspond to or interact with an A valve 220. Similarly, a B valve interface 318 can correspond to or interact with a B valve 218 as shown in FIG. 2C. A proximal air-in-line sensor 322 can be located outside of a cartridge and can interact with a loop or bend in at least partially transparent fluid pathway, for example. In the illustrated example, the sensor 322 is depicted with two vertical portions that can pinch or otherwise be positioned adjacent to a tube running vertically between them. A proximal pressure sensor interface 323 can interact with a pressure sensor 223. A force-sensor, such as resistor 325, can be used to determine whether a cartridge is in physical contact with the hardware, or a portion of a pump having the hardware, shown in FIG. 3A. In some embodiments, an inlet valve 228 is actively driven and can receive actuation from an inlet valve interface 328. Similarly, an outlet valve interface 331 can interact with an outlet valve 231. A plunger 343 can extend toward and interact with a pumping chamber 66 (see FIGS. 2C and 2D). A cassette locator 335 can be used to provide alignment and registration of physical interacting components when a cassette such as shown in FIGS. 2A-2D is inserted into or aligned with the hardware components shown in FIG. 3A. A distal pressure sensor interface 332 is located below a distal air-in-line sensor 336. Above this is located a regulator actuator 367, which can be configured to interact with the precision gravity flow regulator 267.

FIG. 3B illustrates a fluid path through a cassette such as the fluid path shown in the cassette(s) of FIGS. 2A-2D, as actuated by the hardware of FIG. 3A. The physical components of FIGS. 2A-2D and FIG. 3A can control and evaluate fluid in the path illustrated in FIG. 3B. In FIG. 3B, fluid coming in from either a primary line 57A or a secondary line 57B can pass through the A valve 220 or the B valve 218, respectively. Incoming fluid can then mix in a joined passage, and pass a by a proximal air-in-line sensor 322. Fluid can then enter an air trap chamber 59 having a proximal pressure sensor 223. This chamber can allow fluid from two sources to mix. From here, fluid can flow through an inlet valve 228 and from there into a pumping chamber 66. From the pumping chamber 66, fluid can flow through an outlet valve 231, past a distal pressure sensor 232, and past a distal air-in-line sensor 336. Fluid can flow through or pass a precision gravity flow regulator 267 before proceeding from a cassette toward a patient through tubing.

In a system using active, positively-controlled valves with motors, during fluid delivery, the plunger (e.g., 343 in FIGS. 3A and 3C) can repeatedly cycle between the home position and the extended position. To draw fluid into the pumping chamber (e.g., 66) the inlet valve (e.g., 228) is opened. The outlet valve can then promptly close. In some embodiments, opening of the inlet valve can automatically cause the outlet valve (e.g., 231) to close. When the plunger reaches the home position, the plunger motion pauses while the inlet valve (e.g., 228) is closed, pressure is equalized, and the outlet valve (e.g., 231) is opened. Then the plunger extends and the positive pressure forces fluid out of the pumping chamber and into the distal line (e.g., 55) of the set, which can be connected to a patient.

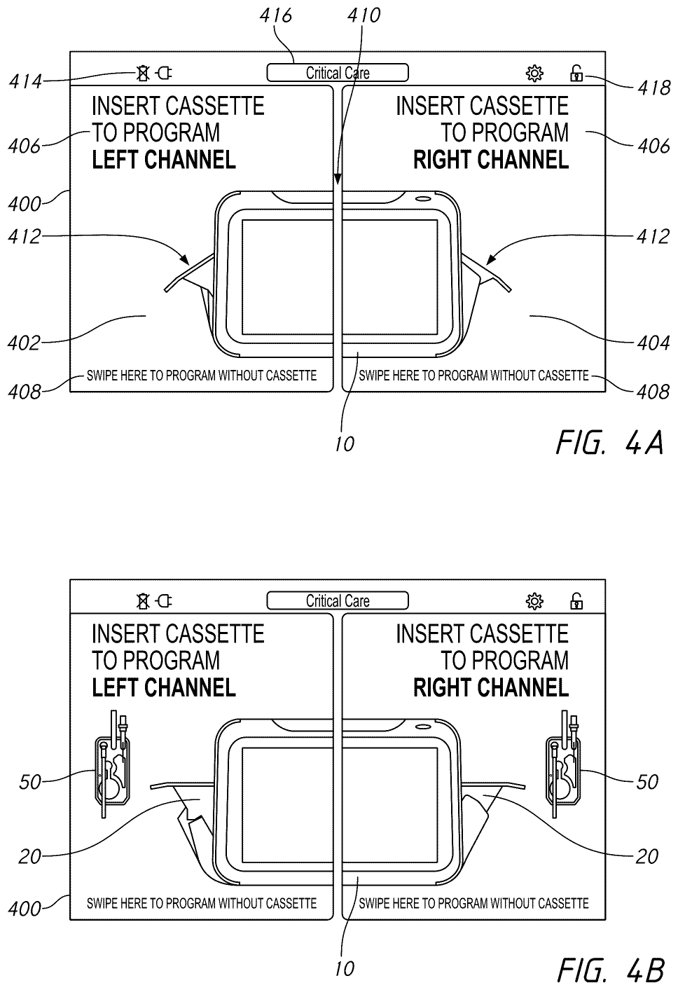

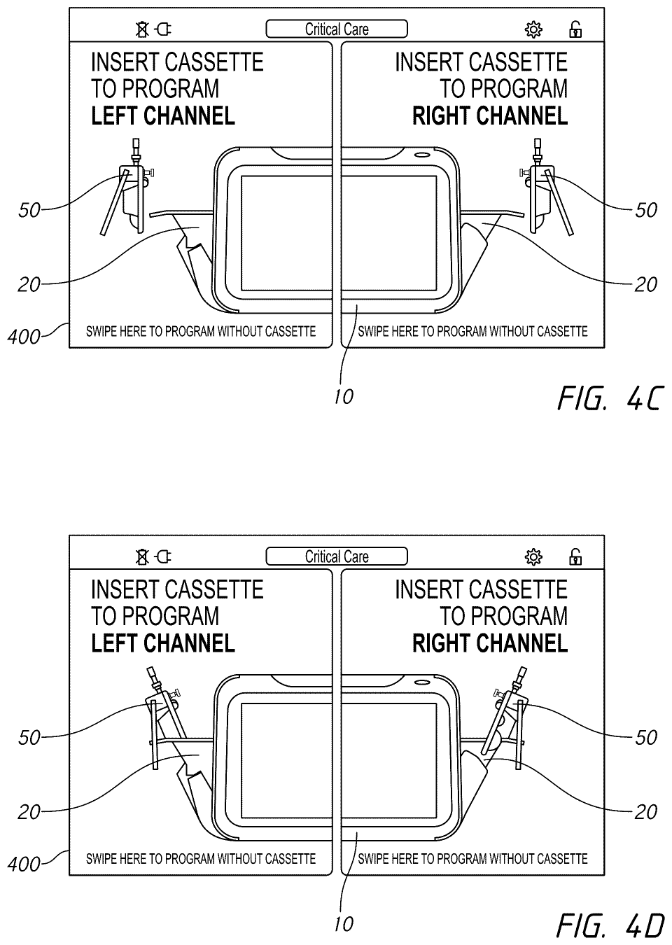

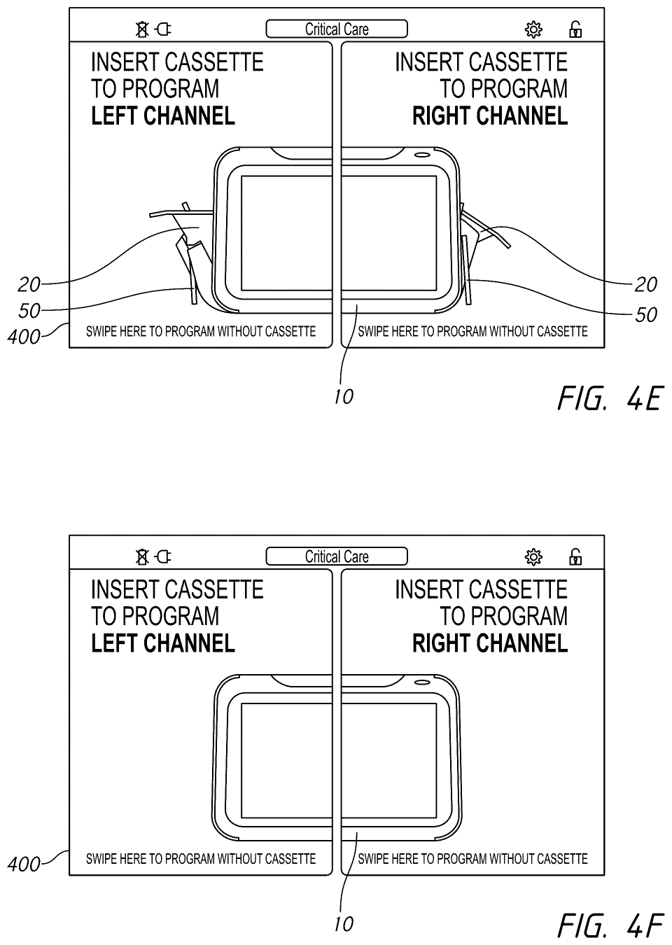

The plunger stepper motor (e.g., motor 342 of FIG. 3C or the motor of FIG. 4C) can be activated by current pulses through the motor windings. In some embodiments, a plunger motor can use different patterns (e.g., 6 different patterns) of pulses can be used, depending on the delivery rate. As the rate increases, a pause between successive steps of the motor decreases. In some embodiments, valve motors can use a single pattern of current pulses through the motor windings. The patterns of current pulses for the motors are advantageously controlled by a PMC microcontroller (e.g., in the controller 380).

FIG. 3C further illustrates schematically how hardware (e.g., FIG. 3A) can interact with a cassette (e.g., FIGS. 2A-2D) along a fluid path. FIG. 3C shows a patient or distal line 55 at the top left corner. At the left is shown an example of a consumable or cassette portion 352. At the right is shown an example of an electromechanical portion 356. In the cassette 352, a distal side 353 is toward the left, and a proximal side 354 is toward the right. A fluid path 351 is illustrated, passing generally from inlets 57A and 57B to outlet 55. Line A 57a leads to a Line A valve or pin 220, which can move to the right and left as shown by the arrow. Similarly, Line B 57B can lead to a Line B valve or pin 218. A spring such as the spring 381 can be deployed with respect to both the valve 218 and the valve 220, and a cam 371 can connect a stepper motor 370 with the valve to 220 and the valve 218. The stepper motor 370 can interact with a line AB position sensor 372, with feedback 373 provided to a controller or controllers 380. A controller 380 can in turn provide input and/or power 374 to the stepper motor 370. In this arrangement, the valves 220 and 218 are actively and positively controlled by a motor and a controller.

For the outlet valve and pin 231 and the inlet valve and pin 228, a stepper motor 377 having a cam 378 and associated springs 382 can interact with the valves 228 and 231. In some embodiments, the cam 371 can cause the associated valves 220, 218 not to be opened simultaneously. In some embodiments, the inlet valves 220 and 218 are not open simultaneously to that fluid does not mix in either of inlet lines 57a or 57b.

Similarly for the cam 378 and the valves 231 and 228, if the cam forms a rigid elongate structure as shown, it can pull on one valve while pushing on the other and when it swings the other direction push and pull in an alternating manner. The valves 228 and 231 can open at alternating times such that fluid intake occurs during a draw portion of a plunger stroke, and fluid is expelled during a push portion of a plunger stroke. Having the valve open simultaneously or other synchronization problems can be avoided to discourage backflow.

An input output valve position sensor 379 can be connected to a physical component of the stepper motor 377. The sensor 379 can provide feedback to the controller or controllers 380, which can in turn send input and/or power 376 to the stepper motor 377.

The controller or controllers 380 can also interact with a third stepper motor 342, which can cause movement of a lead screw 341 connected to a plunger or piston 343, which in turn physically interacts with the pumping chamber 66. A linear position sensor 345 can provide feedback 346 of this process to a controller 380. Similarly, a rotary position sensor 347 can provide feedback 384 to a controller 380. Thus, linear and rotary position feedback can be provided either as a backup, as an alternative, or otherwise. A coupler 344 can be provided between the stepper motor of 342 and the lead screw 341. Input and/or power 385 can be provided from the controller 380 to the stepper motor 342. The plunger or piston 343 can follow a reciprocating pattern as shown by the arrow. Thus, the electromechanical portion 356 of a pump can have multiple reciprocating portions and multiple motors. The reciprocation of the valves 220, 218, 231 and 228 can be timed and coordinated with the reciprocation of the piston 343 (e.g., by controller/s 380) to encourage fluid to move through the fluid path 351. Although additional feedback lines are not shown in FIG. 3C, sensor feedback can be provided from the distal air inline sensor 236 and the proximal area line sensor 222, as well as the distal pressure sensor 232 and the proximal pressure sensor 223.

### Valve Operation

In some modes of operation, the valves 218 and 220 can each be open for some percentage of the duration of an intake stroke of the plunger 343, while the inlet valve 228 is open for approximately the entire duration of the same intake stroke. Concurrent flow can independently control two rates, drawing a proportional amount of fluid from each of lines A and B into the pumping chamber. During an expelling stroke, the outlet valve 231 can remain open approximately the entire time. Intake and expelling strokes can have similar durations. However, an advantageous approach uses a quick intake stroke during which the pump chamber fills, and then a series of smaller output strokes. For example, intake may occur within seconds, while the output strokes continue over a much longer time until the pump chamber needs to be filled again. Proper cadence and sequencing of the motors can be confirmed directly by the feedback from the motors 373, 383, and 385. Proper pressure response of the fluid can be confirmed or measured by the sensors 223 and 232. Potential air bubbles can be evaluated by sensors 222 and 236. System interpretation of sensors 223 and 232, and of 222 and 236, can lead respectively to occlusion alarm and air alarm states that result in unexpected flow discontinuities.

Valve motors such as the motors 370 and 377 of FIG. 3C can be controlled by a pump mechanism controller (“PMC”) microcontroller using a chopper motor drive. The valve motors 370 and 377 can be the same, with one motor used for a pair of valves.

An Inlet/Outlet (I/O) valve motor (e.g., 377 in FIG. 3C) opens and closes the cassette pumping chamber inlet and outlet valves (e.g., 228, 231) in an administration set cassette. The cassette can have a membrane that is exposed by openings in the back of the cassette body above where there are valve chambers in the cassette. The Inlet valve pin (e.g., 228) is opened to allow fluid to enter the pumping chamber (e.g., 66) through the air trap (e.g., 59) from the proximal line, which is selected by the Line A/B Select valves (e.g., 218, 220). When the pumping chamber is filled the Inlet valve (e.g., 228) is closed, the pumping chamber pressure is set and the Outlet valve (e.g., 231) is opened to allow fluid to be pumped into the distal line of the set.

A state machine (e.g., in or associated with the controller 380) can run a program for controlling the I/O valve motor (e.g., 370, 377). In an optical approach, cam flags can protrude from a portion of the drive train. Rotational cam flag signals can be acquired optically during or after each motor step and are monitored using a state machine. As with the other motors, if there is an error in the Inlet/Outlet valve motor position (phase loss), then the motor can be re-initialized to the current position.

The Line A/B Select (LS) valve motor (e.g., 370 in FIG. 3C) opens and closes the Line A and Line B select valves (e.g., 220, 218) in the administration set cassette, using openings in the back of the cassette body for actuator access. The Line A valve (e.g., 220) controls the primary inlet port to the cassette which can be attached permanently to the set proximal tubing. The Line B valve (e.g., 218) controls the secondary inlet port, which may have a screw cap, a Pre-pierced or a Clave attached to it, depending on the type of set.

### Example System Operation

In some embodiments, a pump system can have a cassette door with a handle that supports an administration set cassette such as that illustrated in FIGS. 2A-2D. When the door is open in a loading position the user can slide the cassette into a slot with a cassette guide spring. When the door is closed the cassette is aligned and the front of the cassette makes contact with a door datum surface, actuator and sensor subassemblies (plunger 343 and pins or valves 218, 220, 228, 231) make contact with a cassette elastomeric membrane, and a cassette guide spring can push a fluid shield against the front face of a mechanism chassis. The door can be released from the handle when it is in the loading position, allowing the door to be perpendicular to the mechanism fluid shield. This allows the user to clean the rear of the door and the fluid shield, or to remove any object which has fallen behind the door.

A cassette locator (see, e.g., 335 in FIG. 3A) can be a pin that helps align the cassette with the mechanism as the door is closed and keeps the cassette in the correct position during delivery.

The cassette can have a flow regulator valve (e.g., the precision gravity flow regulator 267, seen in FIG. 2D) distal to the pumping chamber (e.g., the chamber 66 of FIGS. 2A-3D). The flow regulator valve can be closed by the user after an administration set is primed. The proximal line can be clamped as an additional prevention of free flow. As the door is closed, an actuator connected to the door handle can automatically open the flow regulator valve after the pumping chamber outlet valve pin closes the outlet valve. The flow regulator valve can be used by the operator to control fluid flow rate when the administration set is used independently for a gravity drip infusion.

A reciprocating pumping piston/plunger (e.g., the plunger 343 of FIG. 3C) can be actuated by a motor (e.g., the motor 342). As schematically shown in FIG. 3C, a pump plunger motor and drive train can be perpendicular to a pumping chamber membrane opening on the rear of a cassette. The drive train can have location sensors that are monitored by motor control software on a PMC microcontroller (see controller 380 of FIG. 3C). The software can implement state machines which control the motor operation.

An inlet valve to the pumping chamber (e.g., the valve 228) can be actuated by a motor (e.g., the motor 377), and a drive train can extend an actuator through an opening in the rear of the cassette to reach the valve. The same motor can be used for the outlet valve, which can improve synchronization. A default position is with the inlet valve (e.g., the valve 228) closed by a spring (e.g., 382) which can apply steady pressure to a valve pin. The drive train (see generally 377, 378 and related structures) has a location sensor (e.g., 379) that is monitored by (383) motor control software on the PMC microcontroller (e.g., 380). The software implements state machines which can control the motor operation. The same description here generally applies to an outlet valve (e.g., 231), actuated by the same motor (e.g., 377).

Line A select valve (e.g., 220) for primary proximal fluid line A (e.g., 57a) and Line B select valve (e.g., 218) for fluid line B (e.g., 57b) can be actuated by a motor (e.g., 370). As described above for the valves 228 and 231, the valves 220 and 218 can be accessed by a drive train (which may include the cam 371 and springs such as 381) through openings in a cassette, driven by a motor (e.g., 370), as tracked by a location sensor (e.g., 372) and monitored (373) by software in a controller (380).

One or more proximal and distal air-in-line sensors (222, 236) can be used to detect air passage into (proximal) or out of (distal) the cassette. Both sensors can be ultrasound piezoelectric crystal transmitter/receiver pairs. Liquid in the cassette between the transmitter and receiver conducts the ultrasonic signal, while air does not. This can result in a signal change indicating a bubble in the line.

One or more proximal and distal MEMS pressure sensors (223, 232 of FIG. 3C) can be used to detect the pressure of the tubing into (proximal) or out of (distal) the cassette. Microelectromechanical systems (MEMS) pressure sensors are an integrated circuit, which have piezo electric resistors diffused into a micro-machined diaphragm to measure strain from a steel ball that extends through the top of the IC package. The steel ball is driven by a pressure pin which is in contact with the cassette membrane.

A cassette presence sensor detects that the cassette is in the door when it is closed. The sensor can be a dome switch mounted in an infusion mechanism subsystem fluid shield. The dome switch can make contact with the cassette when the cassette is correctly aligned with the fluid shield. The switch output signal can be acquired and processed by PMC microcontroller software (e.g., in controller 380).

Motor control interfaces can provide amplification of control signals output by the PMC microcontroller (e.g., the controller 380). PMC microcontroller software can compute motor winding current values which are converted to analog voltages by a digital-to-analog converter (DAC). The control voltages input to the motor control interface can cause amplifiers to drive the selected motor winding with current modulated by a chopper pulse width modulator controller. Preferably, one motor winding is active at a time.

Sensor interfaces in an infusion mechanism subsystem can convert air-in-line, pressure and motor drive position sensor signals into analog voltage signals. The analog voltages are processed by an analog-to-digital converter (ADC) in the PMC microcontroller which outputs digital values. PMC microcontroller software state machines acquire and process data from the sensors.

Non-volatile memory in an infusion mechanism subsystem can be connected to the PMC microcontroller with a serial communications link (SPI bus). The non-volatile memory can be used to store calibration values for the motor drive trains and sensors during manufacturing. Additional system parameters and an alarm log are also stored by the PMC microcontroller in this memory.

Any control and/or feedback systems of this specification can be configured to generate highly specific, real-time data on how an infusion pump is operating and how fluid in a cassette is responding. This data already exists for precision operation of an infusion device, and it can be conveniently organized and stored (e.g., in a memory of the pump system itself). This data can provide highly accurate predictions of how and when medication will reach a target destination, or achieve a particular level in a target destination. Thus, the sensors, controllers, cam flags, feedback software, etc. described herein is highly valuable in predicting further outcomes, patient medication status, and/or otherwise displaying information to a user.

FIG. 3D is a schematic diagram of some functional components for a medical pump (e.g., the pump 10 of FIGS. 1A-1E) that in some embodiments can be used in connection with the disposable cassette 50 (see FIGS. 2A-D) for delivering a fluid to a patient. Some of the components and/or functions illustrated and/or described in connection with FIG. 3D are alternatives or additions to those illustrated in the cassette of FIGS. 2A-3C. One or more processors or processing units 280 can be included in pump 10 that can perform various operations. The processing unit(s) 280 and all other electrical components within the pump 10 can be powered by a power supply 281, such as one or more components of power source 90 of pump 10. In some embodiments, the processing unit 280a can be configured as a pump motor controller (PMC) to control the electric motor 142 being energized by the power supply 281. When energized, the electric motor 142 can cause the plunger 136 to reciprocate back and forth to periodically actuate, press inward, and/or down-stroke, causing plunger 136 to temporarily press on pumping chamber 66, driving fluid through cassette 50. The motor 142, plunger 136, sensors 128, 290, 132, 140, 266, 144 can be included in or as an integrated part of the pump driver 14 of the pump 10. In some embodiments, as shown, the inlet pressure sensor 128 engages the inlet diaphragm 62 of cassette 50, and the outlet pressure sensor 132 engages the outlet diaphragm 64 of cassette 50. When retracting, moving outward, or on an up-stroke, the plunger 136 can release pressure from pumping chamber 66 and thereby draw fluid from inlet 52 into pumping chamber 66. Differential pressure within the cassette can drive the inlet opening during the pump chamber fill cycle. In some implementations of cassette 50, a flow stop 70 is formed as a pivotal switch in the main body 56 and protrudes a given height from the inner surface 68. This protrusion forms an irregular portion of the inner surface 68 which can be used in some embodiments to align the cassette 50 as well as monitor the orientation of the cassette 50. In some embodiments, one form of a flow stop 70 can provide a manual switch or valve for closing and opening the cassette 50 to fluid flow.

In some embodiments, the processing unit 280a can control a loader 20 of the pump 10 with an electronic actuator 198 and a front carriage being energized by the power supply 281. When energized, the actuator 198 can drive the front carriage 74 between closed or open positions. The front carriage 74 in the open position can be configured to receive the cassette 50 and in the closed position can be configured to temporarily securely retain the cassette 50 until the front carriage is moved to the closed position. A position sensor 266 for the cassette 50 can be provided in the pump 10. The position sensor 266 can monitor the position of a slot 268 formed in a position plate 270. The position sensor 266 can monitor a position of an edge 272 of a position plate 270 within the pump 10. By monitoring the position of the position plate 270, the position sensor 266 can detect the overall position of the front carriage of the loader 20 and/or confirm that the cassette 50 is inserted into the loader 20 of the pump driver 14. The position sensor 266 can be a linear pixel array sensor that continuously tracks the position of the slot 268. Of course, any other devices can be used for the position sensor 266, such as an opto-tachometer sensor.

A memory 284 can communicate with the processing unit 280a and can store program code 286 and data necessary or helpful for the processing unit 280 to receive, determine, calculate, and/or output the operating conditions of pump 10. The processing unit 280a retrieves the program code 286 from memory 284 and applies it to the data received from various sensors and devices of pump 10. The memory 284 and/or program code 286 can be included within or integrally attached to (e.g., on the same circuit board) as the processing unit 280a, which in some embodiments can be the configuration for any processor or processing unit 280 in this specification.

In some embodiments, the program code 286 can control the pump 10 and/or track a history of pump 10 operation details (which may be recorded and/or otherwise affected or modified, e.g., in part by input from sensors such as air sensor 144, position sensor 266, orientation sensor 140, outlet pressure sensor 132, plunger pressure sensor 290, inlet pressure sensor 128, etc.) and store and/or retrieve those details in the memory 284. The program code 286 can use any one or more of these sensors to help identify or diagnose pumping problems, such as air in a pumping line, a pumping obstruction, an empty fluid source, and/or calculate expected infusate arrival time in a patient. The display/input device 200 can receive information from a user regarding a patient, one or more drugs to be infused, and details about a course of infusion into a patient. The display/input device 200 can provide a clinician with any useful information regarding the pumping therapy, such as pumping parameters (e.g., VTBI, remaining volume, infusion rate, time for infusion, elapsed time of infusion, expected infusate arrival time, and/or time for completion of infusion, etc.) Some or all of the information displayed by the display/input device 200 can be based on the operation details and calculations performed by the program code 286.

In some embodiments, the operation details can include information determined by the processing unit 280a. The processing unit 280a can process the data from pump 10 to determine some or all of the following operating conditions: whether or when the cassette 50 has been inserted, whether or when the cassette 50 is correctly oriented, whether or when the cassette 50 is not fully seated to the fixed seat 162, whether or when the front carriage assembly 74 is in an open or closed position, whether or when a jam in the front carriage assembly 74 is detected, whether or when there is proper flow of fluid through the cassette 50 to the patient, and whether or when one or more air bubbles are included in the fluid entering, within, and/or leaving cassette 50. The processing unit 280a can be configured to determine one or more operating conditions to adjust the operation of the pump 10 to address or improve a detected condition. Once the operating condition has been determined, the processing unit 280a can output the operating condition to display 200, activate an indicator window, and/or use the determined operating condition to adjust operation of the pump 10.

For example, the processing unit 280a can receive data from a plunger pressure sensor 290 operatively associated with the plunger 136. The plunger pressure sensor 290 can sense the force on plunger 136 and generate a pressure signal based on this force. The plunger pressure sensor 290 can communicate with the processing unit 280a, sending the pressure signal to the processing unit 280a for use in helping to determine operating conditions of pump 10.

The processing unit 280a can receive an array of one or more items of pressure data sensed from the cassette inner surface 68 determined by the plunger pressure sensor 290 and inlet and outlet pressure sensors 128 and 132. The processing unit 280a can combine the pressure data from the plunger pressure sensor 290 with data from inlet and outlet pressure sensors 128 and 132 to provide a determination as to the correct or incorrect positioning of cassette 50. In normal operation, this array of pressure data falls within an expected range and the processing unit 280a can determine that proper cassette loading has occurred. When the cassette 50 is incorrectly oriented (e.g., backwards or upside down) or when the cassette 50 is not fully seated to the fixed seat 162, one or more parameters or data of the array of pressure data falls outside the expected range and the processing unit 280a determines that improper cassette loading has occurred.

As shown, in some embodiments, the processing unit 280a can receive data from one or more air sensors 144 in communication with outlet tube 55 attached to the cassette outlet 54. An air sensor 144 can be an ultrasonic sensor configured to measure or detect air or an amount of air in or adjacent to the outlet 54 or outlet tube 55. In normal operation, this air content data falls within an expected range, and the processing unit 280a can determine that proper fluid flow is in progress. When the air content data falls outside the expected range, the processing unit 280a can determine that improper air content is being delivered to the patient.

Processing unit 280a can continuously or periodically communicate with an independent and separate processor or processing unit 280b to communicate information to the user and/or to receive data from the user that may affect pumping conditions or parameters. For example, processing unit 280a can communicate by wire or wirelessly with processing unit 280b which can be configured as a user interface processor or controller (UIC) to control the output and input of display/input device 200, including by displaying an operating condition and/or activate indicator 18 to communicate with a user. In some embodiments, processing unit 280b can receive user input regarding pumping conditions or parameters, provide drug library and drug compatibility information, alert a user to a problem or a pumping condition, provide an alarm, provide a message to a user (e.g., instructing a user to check the line or attach more fluid), and/or receive and communication information that modifies or halts operation of the pump 10.

An independent and separate processor or processing unit 280c can be configured as a communications engine (CE) for the pump, a pump communications driver, a pump communications module, and/or a pump communications processor. Processing unit 280c can continuously or periodically communicate with processing units 280a and 280b to transmit and/or receive information to and from electronic sources or destinations separate from, outside of, and/or remote from, the pump 10. As shown, processing unit 280c can be in electronic communication with or include a memory 284 and program code 286, and processing unit 280c can be in communication with and control data flow to and from a communicator 283 which can be configured to communicate, wired or wirelessly, with another electronic entity that it separate from the pump 10, such as a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The communicator 283 can be or can comprise one or more of a wire, a bus, a receiver, a transmitter, a transceiver, a modem, a codec, an antenna, a buffer, a multiplexer, a network interface, a router, and/or a hub, etc. The communicator 283 can communicate with another electronic entity in any suitable manner, such as by wire, short-range wireless protocol (Wi-Fi, Bluetooth, ZigBee, etc.), fiber optic cable, cellular data, satellite transmission, and/or any other appropriate electronic medium.

As shown schematically in FIG. 3, a pump 10 can be provided with many components to accomplish controlled pumping of medical fluid from one or more medical fluid sources to a patient. For example, one or more processors or processing units 280 can receive various data useful for the processing unit(s) 280 to calculate and output the operating conditions of pump 10. The processing unit(s) 280 can retrieve the program code 286 from memory 284 and apply it to the data received from various sensors and devices of pump 10, and generate output(s). The output(s) are used to communicate to the user by the processing unit 280b, to activate and regulate the pump driver by the processing unit 280a, and to communicate with other electronic devices using processing unit 280c.

### Additional Features

In some embodiments, the pump 10 can be provided with an internal computer program code 286 included within memory 284 in electronic communication with, or within, on, and/or otherwise part of, the processing unit 280B of the UIC to control the output and input of display/input device 200. As shown in FIGS. 4A-9, the internal computer program code 286 can include steps, instructions, algorithms, and/or data configured to provide a text and/or graphical display 400 to provide information to and receive input from a user.

As shown in FIG. 4A, the display 400 on the display/input device 200 can comprise multiple display and/or input regions, such as a first region 402 and a second region 404. The first and second regions 402, 404 can be spatially separated from each other in a meaningful way that communicates useful information to a user. For example, the first region 402 can be located on the left side of the display 400 so that it is closest to the pump driver 14 on the left side of the pump 10, and the second region 404 can be located on the right side of the display 400 so that it is closest to the pump driver 14 on the right side of the pump 10. The first region 402 can be configured to receive and/or display relevant information about the left pump driver 14, and the second region 404 can be configured to receive and/or display relevant information about the right pump driver 14. Positioning each region 402, 404 of the display device 200 closest to the pump driver 14 as to which it receives and/or displays information enables the user to readily recognize which data entry or information display corresponds to which physical cassette(s) 50 and/or fluid source(s) (e.g., one or more IV bags or vials). As shown, text can be provided to communicate or emphasize to the user the pump driver 14 that each region 404, 404 controls, such as “Left Channel” and “Right Channel.”

The left and/or right indicators 18 can be controlled by the processing unit 280B of the UIC to selectively illuminate, such as to illuminate to indicate that instructions are being provided or information is being received regarding the pump driver 14 adjacent to such illuminated indicator 18 and/or that such pump driver 14 is actively pumping medical fluid from a fluid source toward a patient. The left and right indicators 18 can be controlled by the processing unit 280B to communicate additional or different information, such as by selectively illuminating in multiple colors and/or by flashing to indicate an operational state (e.g., green and/or steady light) or a warning or disabled state (e.g., red and/or flashing light).

As illustrated, in some embodiments, when a cassette 50 has not been properly installed into one or more of the pump drivers 14, one or more position sensors in the pump driver 14 without the cassette 50 can detect the absence of the cassette 50 and communicate this information to the processing unit 280B, which can then cause display 400 to communicate an instruction 406 to the user through the first and/or second regions 402, 404 that notifies the user that no cassette 50 is currently inserted, that requests that the user insert a cassette 50 into the pump, and/or that disables the entering of information and/or that disables programming a course of infusion for such pump driver 14 unless or until the cassette 50 is properly inserted. For example, in some embodiments, the first and/or second regions 402, 404 can display text with such an instruction 406 or notice (e.g., as shown, “Insert Cassette to Program” Left and/or Right “Channel”).

In some embodiments, as shown, the processing unit 280B can be configured to access from the electronic memory 284 in communication with the processing unit 280B one or a plurality of images comprising a display with moving graphics and/or an animation 410 to help notify or instruct the user that a cassette 50 needs to be inserted into the loader 20 of the pump driver 14 and/or to show how to insert the cassette 50 into the loader 20 of the pump driver 14. For example, as illustrated in FIG. 4A, a graphic and/or an animation 410 can comprise a schematic illustration of the pump 10 and/or cassette 50. In some embodiments, when a cassette 50 is not inserted into a respective pump driver 14, the graphic and/or animation 410 can illustrate the pump driver 14 in a state without the cassette 50. For example, as shown, the graphic and/or animation 410 can illustrate a portion of the loader 20 of the pump 10 in an open and/or extended position 412.

As shown in a comparison between FIGS. 4A-4F, a continuously looped, repeating sequence of changing graphics and/or an animation 410 can provide an engaging and effective way of notifying and/or instructing a user to insert the cassette 50 into the loader 20 of the pump driver 14 in the proper location before use. For example, the changing graphics and/or the animation 410 can comprise a schematic representation of the cassette 50 initially spaced away from the pump 10 (e.g., FIG. 4B), later brought near to the respective loader 20 of the pump driver 14 (e.g., FIG. 4C), and then inserted into the loader 20 of the pump driver 14 (e.g., FIGS. 4D and 4E). The pump 10 can then be shown with the loader 20 in a closed or retracted position (e.g., FIG. 4F). The animated motion of the cassette 50 and the loader 20 of the pump driver 14 can be shown to schematically repeatedly change from an open and/or extended position to a closed and/or contracted position, as shown, thereby urging the user to insert the cassette 50 into the loader 20 of the pump 10. The one or more position sensors in the pump driver 14 can detect when the user properly inserts the cassette 50 into the pump 10 and communicate a signal to the processing unit 280B of the UIC, causing the UIC to automatically change the display 400 to cease showing the graphic and/or animation 410 and automatically proceed to a screen on the display 400 where a user can input and/or view pumping information and/or parameters. In displays 400 that include multiple regions 402, 404, as shown, the processing unit 280B can cause the UIC to change the display only in the region 402, 404 corresponding to the pump driver 14 in which the cassette 50 has been inserted, leaving the other region 402, 404 to continue displaying all or part (as shown) of the graphic and/or animation 410 urging the user to insert the cassette 50 into the other pump driver 14 corresponding to the other region 402, 404. After or when the user inserts the cassette 50 into either loader 20 of either pump driver 14, the processing unit 280B can automatically transition the display 400 corresponding to that pump driver 14 to a screen permitting the user to make one or more selections relating to inputting and/or confirming information regarding a patient, a medical fluid to be infused, and/or parameters relating to a course of infusion, as illustrated in FIG. 5.

As shown in FIG. 4A, either or both of the regions 402, 404 of the display 400 can provide an instruction 408 or notice about the absence of the cassette 50, and/or can provide an input location and/or another way for the user to override the instruction 408 or notice about the absence of the cassette 50 and then permit the user to proceed to enter and/or program information into the region 402, 404 corresponding to a respective pump driver 14. For example, as shown, the region 402, 404 can display an overriding message indicating “Swipe Here to Program Without Cassette” or any similar message, and that area of the region 402, 404 can be configured to receive a swiping or other user input to permit entering information or programming without a cassette 50. In most situations, the infusion of fluid cannot begin without a cassette 50 inserted into the pump 10, but once programmed the pump 10 stands ready for infusion to begin immediately upon insertion of the cassette 50 if the user has overridden the instruction 406 or notice and previously entered pumping information before insertion of the cassette 50 into the pump 10.

This initial stage of use or initial screen and/or other screens for the display 400 can include one or more other items or features to convey useful information to a user and/or to receive input from a user. For example, as shown, the display 400 can communicate to the user information about the electrical power source of the pump 10, using a power indicator 414. For example, the power indicator 414 can inform the user whether the pump 10 is in electrical communication with an external power source. In the example shown, the display 400 is communicating to the user that the battery 94 of the pump 10 does not have sufficient electrical power to operate the pump 10 (or is not attached) and an external power source is connected to the pump 10. The display 400 can alternatively be configured to display a message and/or graphic indicating that the pump 10 is not attached to an external power source and is operating using the electrical power from the onboard battery 94, or that the pump 10 is attached and capable of receiving electrical power from both the battery 94 and an external power source. The power indicator can in some embodiments show numerically and/or graphically how much electrical power remains in the battery 94.

The display 400 can provide information communicating to the user a mission message 416 showing a temporarily and selectively changeable assigned purpose, location, department, owner, and/or task for the pump 10. For example, as illustrated, the mission message 416 indicates “Critical Care,” demonstrating that the pump 10 is temporarily assigned for use in a critical care department of a hospital. The mission message 416 can be inputted by a local user of the pump 10 and/or can be inputted remotely by a user and/or a computer system in communication with the pump 10 through the communicator 283 in communication with the processing unit 280C of the CE. In some embodiments, the pump 10 can include a position or location sensor, such as a GPS sensor, an NFC/RFID device, and/or a wired or wireless (e.g., WiFi-enabled) sensor, that is configured automatically to determine the location of the pump 10 and/or automatically to display and/or change, without input from a local user, the mission message 416 to reflect the purpose, location, department, owner, and/or task for the pump 10, as correlated or inferred from its location. For example, the mission message 416 can be configured to automatically display as “Critical Care” when the pump 10 is powered up or activated in the location of the critical care department of the hospital and/or to change from displaying one location (e.g., “Critical Care”) to displaying another location (e.g., “Pediatric”) when the pump 10 is moved from one location of the hospital (e.g., the critical care location) to another location of the hospital (e.g., the pediatric location). The display 400 can include a security indicator 418 showing whether the pump 10 is in a locked state (e.g., prevented from providing and/or receiving one or more types or all information and/or instructions from a user), or an unlocked state (e.g., permitted to provide and/or receive one or more types or all instructions and/or instructions from a user).

As illustrated in FIG. 5, the display 400 can prompt a user to input information in the first and second regions 402, 404 for separate pump drivers 14. The type of information shown and received in the respective first and second regions 402, 404 can be different for each one. For example, if a cassette 50 has not been inserted into one of the first and second pump drivers 14, the respective corresponding first or second region 402, 404 can remain as shown in FIGS. 4A-4F, while at the same time if a cassette 50 has been inserted into the other of the first and second pump drivers 14, the respective corresponding other first or second region 402, 404 can automatically transition to the screen shown in that region in FIG. 5. As shown, the screens displayed in the first and second regions 402, 404 can be split, operating in different stages from each other, depending on their different circumstances.

In some embodiments, as shown, each of the pump drivers 14 and cassettes 50 can be configured to receive and/or be coupled with multiple sources of medical fluid. For example, the left pump driver 14 can be configured to receive and/or be coupled with a primary and a secondary line or tube of incoming medical fluid from at least two medical fluid sources, and the right pump driver 14 can be configured to receive and/or be coupled with a primary and a secondary line or tube of incoming medical fluid from at least two medical fluid sources. Each of the pump drivers 14 can intermittently, alternatively, generally continuously, and/or generally simultaneously deliver multiple fluid sources to a patient. The capability of receiving and conveying to a patient a plurality of fluid sources through a single pump driver and cassette is described and illustrated in U.S. Pat. No. 4,842,584, previously incorporated by reference in its entirety in this application, and any structure, material, function, method, or step that is described and/or illustrated in that patent for doing so can be used with or instead of any structure, material, function, method, or step that is described and/or illustrated in this specification.

As shown in FIG. 5, the processing unit 280B can retrieve from its memory 284 and display on the display/input device 200 a graphical user interface that is configured to permit a user of the pump 10 to select to input and/or view pumping information from at least a first pump driver 14 and cassette 50 represented in the first region 402, and a second pump driver 14 and cassette 50 represented in the second region 402. In each of the first and second regions 402, 404, an association graphic 420 can help associate in a user's mind the correlation between the user's selection and the respective pump driver 14 and cassette 50 to which it applies. For example, the association graphic 420 can comprise an arrow as shown, and/or any other spatial and/or directional indicator (e.g., a line, a circle, a triangle, etc.), to denote and/or to point the user in the direction of the pump driver 14 and cassette 50 as to which the selection applies. Each of the first and second regions 402, 404 can include association text 422 describing the location and/or other identifier of the corresponding pump driver 14 and/or cassette. For example, in some embodiments as shown, the association text 422 can specify “Left Cassette” and/or “Right Cassette.”

Each of the first and second regions 402, 404 can include a product graphic 424 that schematically illustrates one or more physical products or portions thereof in each respective pumping line, such as a cassette 50 and/or tubing as shown, to help show and/or remind the user how information to be inputted or viewed relates to the physical pump driver 14 and cassette 50 connections made by the user on the pump 10. The product graphic 424 can include one or more depictions of shapes and/or properties of the cassette 50, fluid source, one or more fluid line components, and/or a pump driver 14, etc. For example, as shown, the product graphic 424 in some embodiments can include one or more depictions of the cassette 50, tubing, a drip chamber, a needle-free connector, and/or a patient output line, etc. The product graphic 424 can include one or more connection points 426, 428 to help associate and/or correlate the region of a user's selection with the corresponding physical configuration of the tubing and/or connections or fluid communication between the cassette 50 and one or more medical fluid sources. Any portion or region of the display 400 can be configured as a sensing region that is capable of detecting a user's touch selection in such region and/or generating an electronic signal transmitted to the processing unit 280B to indicate a user selection relating to that region. For example, as illustrated, a primary connection point 426 can illustrate that the information to be inputted or viewed when a user selects and/or touches a first sub-region 430 (e.g., “Left Primary Line—L1” or “Right Primary Line—R1”) will affect and/or display pumping parameters on the illustrated one of a plurality of lines of the physical cassette 50 that touches (as shown), is within, is near, and/or is adjacent to, this sub-region 430 on the display 400 at the primary connection point 426. A secondary connection point 428 can illustrate that the information to be inputted or viewed when a user selects and/or touches a second sub-region 432 (e.g., “Left Secondary Line—L2” or “Right Secondary Line—R2”) will affect and/or display pumping parameters on the illustrated one of a plurality of lines of the physical cassette 50 that touches (as shown), is within, is near, and/or is adjacent to, this sub-region 432 on the display 400 at the secondary connection point 428. When a user touches any of the sub-regions 430, 432, the display/input device 200 is configured to convey an electrical signal to the processing unit 280B which is configured to change the screen by retrieving instructions and/or data from its memory 284 to permit input and/or viewing of selected pumping data and/or parameters.

In some embodiments, as illustrated, one or more machine-readable codes 434 can be provided on the display 400 by the processing unit 280B to help coordinate information exchange between or among different computer systems. For example, as illustrated, a combination of dark and light regions (e.g., a QR code or a bar code) can encode information that can be read by an optical reader of another computer system that is aimed at and captures information from one or more of the machine-readable codes 434 on the display 400. The encoded information from the one or more machine-readable codes 434 can itself convey information to the other computer system about the configuration of the pump 10 and/or any or all related components (e.g., the cassette 50, tubing, etc.), and/or the encoded information can create a link of identifying information between or among one or more computer systems that can permit separate and independent communication of information through a different communication channel enabled by the link between or among such computer systems, using processing unit 280C and communicator 283.

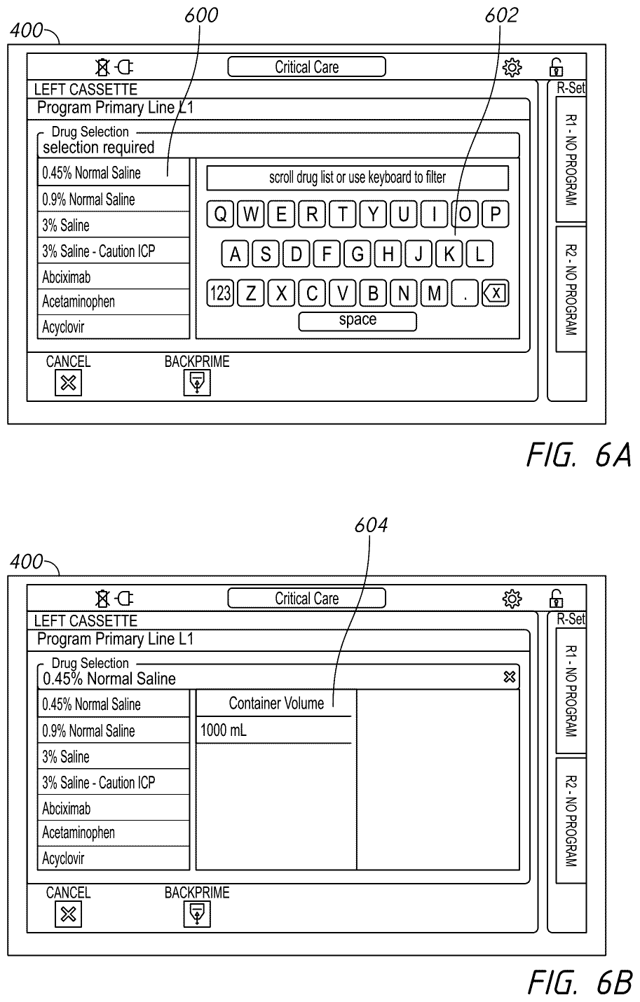

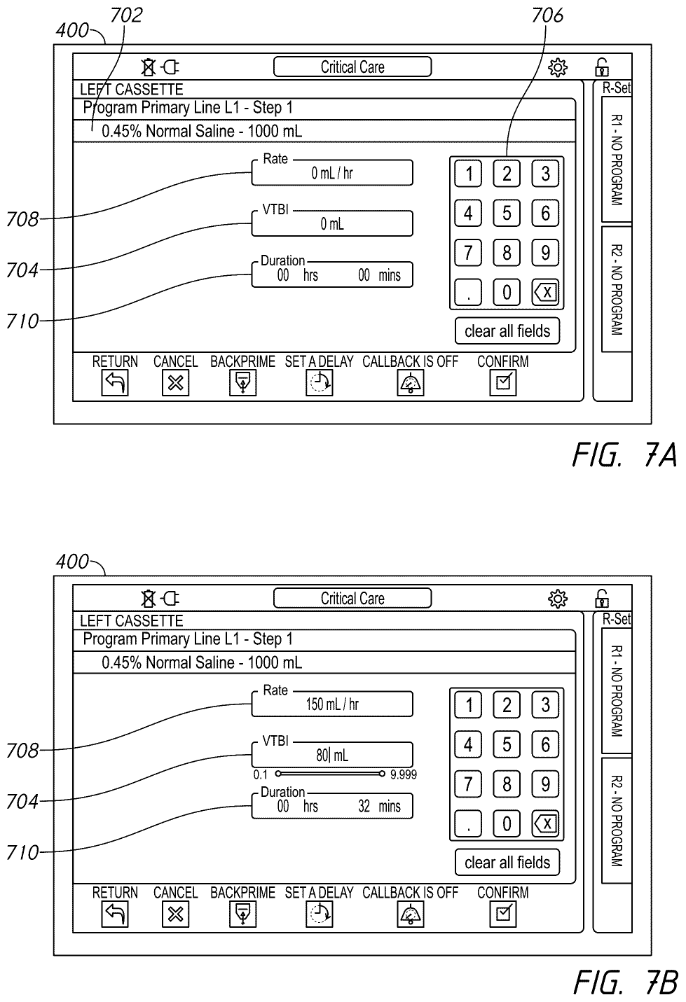

As shown in FIGS. 6A-9, when a user selects and/or is directed into inputting and/or programming a particular pump driver 14, the processing unit 280B can retrieve from its memory 284 and/or its program code 286 information and/or data to enable the display/input device 200 to receive from the user and/or display to the user information relating to a course of pump infusion for that pump driver 14.

For example, as illustrated, when a user touches sub-region 430 of the display 400 shown in FIG. 5, the display 400 can transition to a user input phase as in the example depicted in FIG. 6A. If a user instead touches another region or sub-region, the display 400 can be configured to transition to a user input phase specific to the information, prompts, and/or graphics depicted in that region or sub-region, such as for a different pump driver 14.

As shown in FIG. 6A, in some embodiments, the display 400 can be configured to provide a user interface that requests the user to input or otherwise identify the drug to be infused into the patient through the pump driver 14 associated with the user's choice. For example, the display 400 can provide a scrollable or otherwise selectable list of a plurality of possible drug choices in one region of the display 400 and/or the display 400 can permit the user to input a drug choice using a keyboard, such as a virtual touch-screen keyboard 602 as shown. Any other suitable input mode, such as any used in any embodiment anywhere in this specification, can be used to receive information or one or more selections from a user, such as a series of buttons, a physical keyboard, a mobile electronic device in electronic communication with the pump 10, a microphone in electronic communication with a voice-recognition system within or in electronic communication with the pump 10, and/or a camera capable of viewing one or more gestures from the user, etc. As shown, the selectable list of possible drug choices can include one or more additional data items regarding a drug, such as the concentration of the drug and/or one or more constituents of the drug, and/or information or warnings regarding the drug.

The display 400 can include a user prompt or input 604 and/or can be configured to receive an input or selection from the user of one or more other items of information regarding a particular course of medical fluid infusion (instead of or in addition to the drug selection as shown in FIG. 6A). For example, as illustrated in FIG. 6B, the display 400 can be configured to prompt and/or receive an input from the user regarding one or more features of a drug to be infused, such as the total volume of the drug contained within the medical fluid source that is attached to the cartridge 50 coupled to the pump driver 14 associated with this display stage, as shown. In some embodiments, the user can be prompted to input or select the concentration of the drug, the manufacturer of the drug, one or more variants of the drug, and/or the date of manufacture of the drug, etc. As shown, some commonly used or suggested possibilities can be provided as defaults, such as in a drop-down selection region or in any other way, or the user can be permitted to enter one or more values, including values that may be different from those suggested.

The display 400 can be configured to permit the user to specify or pre-program multiple steps in a course of infusion with one or more pumping parameters or variables that can automatically change when a pre-determined time elapses or when some other aspect of the pumping course has been accomplished, such as the pumping of a pre-determined volume of fluid, without requiring the user to return to the pump 10 to change the pumping parameters. For example, a user can indicate that a first step can proceed at a higher infusion rate and then transition to a second step at a lower infusion rate. Each step can be configured to last for a user-specified amount of time. As shown in FIG. 7A, the display 400 can be configured to permit the user to enter a single, unchanging pumping course or to permit a user to enter multiple, sequential, and/or consecutive steps in a pumping course, with each different step changing at least one pumping parameter.

As illustrated in FIG. 7A, in some embodiments, the display 400 can be configured to prompt a user to input a plurality of items of information relating to how the medical fluid will be infused into the patient, and/or the display 400 can be configured to receive at least one item of information relating to how the medical fluid will be infused into the patient and/or either of the processors 280A or 280B can be configured to provide one or more defaults of pumping information or parameters, and/or can be configured to calculate and/or to derive one or more other items of pumping information or parameters from or relating to one or more inputs or selections made by the user.

For example, in some embodiments (not shown), the volume-to-be-infused (VTBI) into the patient can be auto-populated or initially set as a changeable default that is equal to the total volume of fluid that is contained within the fluid source container (e.g., as either previously inputted by the user, or as communicated electronically separately to the pump 10, and/or as calculated or derived by the pump 10), or that is equal to some pre-determined proportion or fraction of the total volume of fluid that is contained within the fluid source container (e.g., 90% of the total volume of fluid that is contained within the fluid source container).

As another example, in some embodiments such as is shown in FIG. 7B, certain parameters can be calculated and/or derived by the processor 280B from inputs of one or more other parameters, such as calculating and/or deriving the pumping duration time from the pumping rate and the VTBI. In the example shown in FIG. 7A, the display 400 can be configured to provide a user input (e.g., a touch-screen keyboard 706 on the display 400, as shown) to allow the user to input a first value, such as the rate at which the fluid from the fluid source container will be pumped by the pump driver 14 into the user (e.g., in volume, such as milliliters, within a specified amount of time, such as hours), which can be shown on the display 400 in a rate display 708. The display 400 can be configured to allow the user to input a second value, such as the VTBI (e.g., in volume, such as milliliters), which can be shown on the display 400 in a volume display 704. As illustrated, when one or more pumping values (e.g., first and second values) are provided or set (e.g., rate and volume), the processor 280B can derive and/or calculate another pumping value (e.g., a third pumping value), such as the time for such volume to be pumped at the specified rate, which can then be displayed in the infusion duration display 710. In the example shown in FIG. 7B, in the processor 280B, the volume-to-be-infused of 80 mL (see volume display 704) is divided by the pumping rate of 150 mL/hr (see rate display 708) and then converted from hours to minutes, yielding a pumping duration of 32 minutes (see infusion duration display 710). Any other possible calculation and/or derivation can be used. As illustrated, the display 400 can include a selected drug indicator 702 to confirm to the user the name drug that was selected by the user previously. As illustrated, the display can show one or more hard or soft limits for certain pumping parameters. For example, as shown in FIG. 7B, a limit indicator (e.g., a bar and/or upper and lower values, as shown) is provided that shows a potential range for the VTBI. In some embodiments, no values outside of this range will be accepted (hard limit); and in some embodiments, values outside of this range will be accepted but a notice will be given to the user that the parameter is outside of the expected range (soft limit). A limit indicator can be provided for any pumping value, whether inputted by the user or received from a memory or from a remote source or calculated by the processor 280B. In some embodiments, such as where the infused fluid has a time constraint or requirement, the limit indicator can be provided on the infusion rate; and in some embodiments, such as where the infused fluid is intermittent or otherwise not time-constrained, the limit indicator can be provided on the VTBI, the duration, and/or the rate.

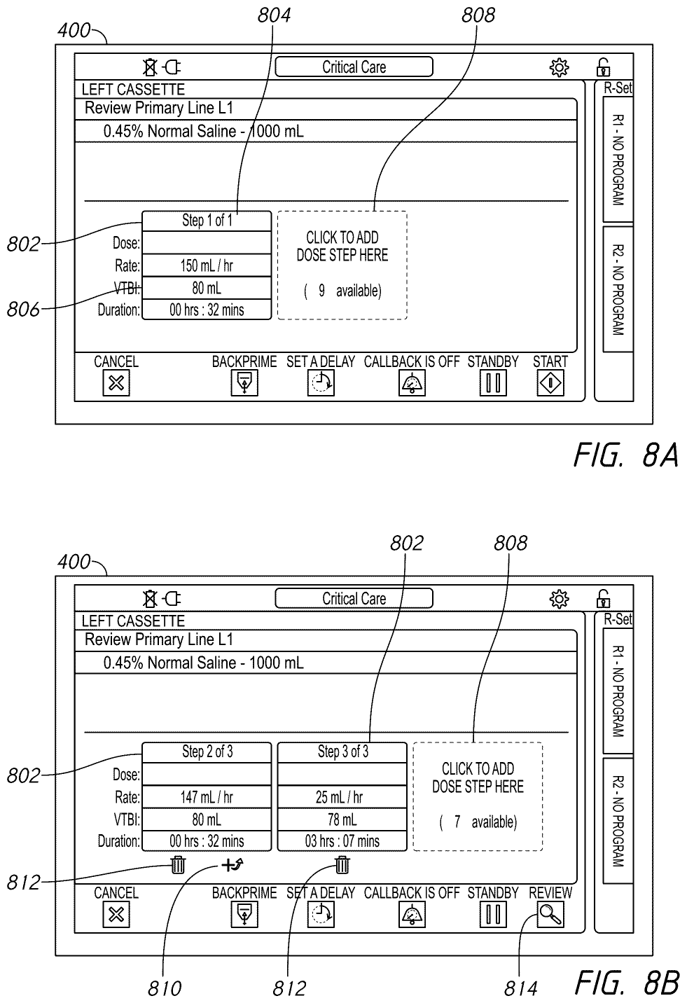

FIG. 8A illustrates that the programming or pumping parameters entered, confirmed, and/or set by the users in a particular stage or step, such as in the manner illustrated in FIGS. 7A-7B, and/or otherwise calculated and/or derived by and/or communicated to the pump 10 (for example, through wired or wireless electronic communication, e.g., using communicator 283 and/or processing unit 280C), can be shown on the display 400 in a summarized, distinct, discrete, encapsulated, separated, and/or grouped way. For instance, the display 400 can provide an infusion parameter grouping 802 that describes and/or represents at least one step or stage in an infusion course for a patient. The parameter grouping 802 can include a grouping label 804 that can identify the designated pumping parameters in any suitable way and/or describe how the parameter grouping 802 fits in with or is ordered in relation to other parameter groupings (see FIG. 8B), such as stating “Step 1 of 1.” The parameter grouping 802 can display one or more pumping values or parameters 806. In the example shown, the parameter grouping 802 can include a boundary and can comprise a designated shape that is common to and/or generally the same as one or more additional parameter groupings, such as a generally square shape (as shown), a generally rectangular shape, a generally circular shape, etc. The display 400 can be configured to include an adding sub-region 808 that is configured to permit the user to specify one or more additional parameter groupings 802 that will be added and/or inserted after (or before) the one or more parameter groupings 802 already shown on the display 400. When a user actuates (such as through the touch screen) the adding sub-region 808, the display 400 can be configured to move to and/or return to a mode for receiving pumping parameters or values, such as shown in FIGS. 7A-7B, for that additional parameter grouping 802. One or more additional parameter grouping 802 can then be shown sequentially on the display 400 in a manner that represents the order of execution of the parameter groupings 802 during pumping.

As shown in FIG. 8B, when a plurality of parameter groupings 802 are provided, each parameter grouping 802 can be shaped and can be sufficiently small in size such that multiple parameter groupings 802 can be viewed on the display 400 at the same time and/or can be sufficiently large to enable viewing of the details within each parameter grouping 802 by an average user without difficulty. In some embodiments, as shown in FIG. 8B, at least three parameter groupings 802 can be shown on the display 400 at the same time. More parameter groupings 802 can be programmed in and/or designated by a user, such as at least 8 or at least 10 parameter groupings 802. When not all parameter groupings 802 are shown on the display 400 at the same time, the display 400 can be configured to allow the user to select individual parameter groupings 802 or subsets of parameter groupings 802 for viewing, such as by scrolling horizontally and/or vertically through the parameter groupings 802 (e.g., by swiping back and forth and/or up and down on the touch screen on which the display 400 is shown).

Parameter groupings 802 can be edited as needed or desired before commencing a course of infusion and/or during a course of infusion (for parameter groupings 802 not yet executed). For example, as shown in FIG. 8B, a new parameter grouping 802 can be added for execution between two existing parameter groupings 802, such as by touching an addition icon 810 which can cause the display 400 to return to a mode for receiving pumping parameters or values, such as shown in FIGS. 7A-7B, for that additional parameter grouping 802, and which can cause that additional parameter grouping 802 to be positioned afterward on the display 400 between the two parameter groupings 802 where the addition icon 810 was located when touched. Any parameter grouping 802 can be deleted by a user by touching on a deletion icon 812 adjacent to and/or associated with a particular parameter grouping 802. When each of the desired parameter groupings 802 have been entered and/or set, a user can actuate the review icon 814 which can permit the user to recheck the accuracy and/or correctness of the pumping values and/or parameters in each parameter grouping 802, at which point the review icon 814 can change to a start icon (not shown). When the user actuates the start icon, the infusion of medical fluid can begin sequentially through and/or in accordance with each of the multiple pumping stages as specified in each consecutive parameter grouping 802. In some embodiments, as shown, the user is not permitted to start infusion until the user has first actuated the review icon 814 and/or the user has first caused each of the pumping stages in each parameter grouping 802 to appear on the screen to enable review by the user. The same or similar steps and/or graphical user interfaces can be utilized to input pumping values for any or all of the pump drivers 14, any or all of the input tubes 57 of the cassette 50 from different medical fluid sources, and/or any or all of the sub-regions 430, 432 (see, e.g., FIG. 5).

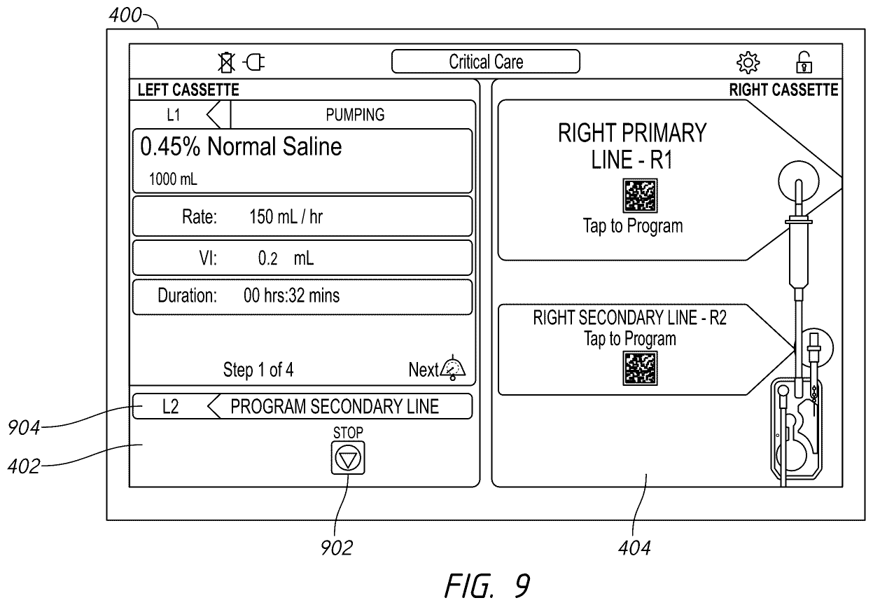

As illustrated in FIG. 9, when a previously programmed course of medical fluid infusion is commenced on a pump driver 14, the display 400 can show that such pump driver 14 is active and pumping, with a designation and/or description of the real-time pumping parameters, separate from and/or independent of the state of one or more other pump drivers 14. For example, as shown, the first region 402 can show that a first pump driver 14 (e.g., on the left) is active and pumping, while the second region 404 can show that a second pump driver 14 (e.g., on the right) is not active and is not pumping and that the second region 404 can be actuated (e.g., by touching) to enable programming of the second pump driver 14, such as is shown in FIG. 5. The pumping can be immediately stopped by actuating the stop icon 902. An additional (e.g., secondary) line for pump driver 14 corresponding to an additional (e.g., secondary) input tube 57 and/or another pump driver 14 (e.g., on the right) can be programmed by touching respectively in a sub-region 904 designated for such additional line and/or in a region or sub-region designated for such other pump driver 14, using any appropriate display mode, data input, communication, calculation, and/or derivation method or step, including but not limited to any or all of those illustrated and described in connection with FIGS. 5-8B.

In some embodiments, it is desirable to lock the display 400 from some or all user input to resist inadvertent contact by a user, a patient, medical equipment, and/or any other contact that could unintentionally be treated as an input of some kind by the pump 10. For example, the processor 280B can auto-lock the display 400, rendering it unresponsive to most or all touch contact after a predetermined period of time following a user touch of the screen, such as at least about 20 seconds and/or less than or equal to about 40 seconds. The processor 280B can auto-lock the display 400 in any other suitable situation when the risk of inadvertent screen contact is high, such as: when one or more motion and/or location sensors in the pump 10 (e.g., a GPS sensor, an accelerometer, a WiFi locator, an acoustic sensor, an infrared sensor, etc.) detect that the pump 10 is being moved; when the electrical cable 92 is removed from an electrical outlet and the pump 10 transitions to receiving only electrical power from its onboard battery 94 (which may suggest that the pump 10 is about to be moved); and/or when a series of touches, movements, and/or other contact on the screen indicate by their nature (e.g. repetition, extended length of contact, and/or undecipherable meaning) that unintentional input is likely, such as when a person is cleaning the screen, a person is leaning against or holding the screen for support, or a child is playing with the screen, etc.

After the display 400 moves into an auto-lock mode, the processor 280B can become unresponsive to all but a certain type of pre-determined touch input. For example, in an auto-lock mode, the display 400 may present the screen as normal until it is touched in some manner and then it may display an activation icon such as with a “swipe to unlock” message or other icon or message prompting a user to perform a particular type of pre-determined or intentional touch or motion on or across a certain portion of the screen in order to reactivate the display 400 to receive standard inputs from a user. In some embodiments, the activation icon can appear at essentially the same time as the display enters auto-lock mode. In some embodiments, the display 400 can be configured to enter a lock mode when intentionally prompted by a user to do so (such as by actuating a lock icon on the display 400 or a lock button on the housing of the pump 10), rather than triggering the lock mode automatically. The return from an intentional lock mode to a normal operating mode can be the same as or similar to that described for returning from an auto-lock mode to a normal operating mode.

### Terminology and Conclusion

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in this description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single disclosed embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Terms of equality and inequality (e.g., less than, greater than) are used herein as commonly used in the field, e.g., accounting for uncertainties present in measurement and control systems. Thus, such terms can be read as approximately equal, approximate less than, and/or approximately greater than. In other aspects of the invention, an acceptable threshold of deviation or hysteresis can be established by the pump manufacturer, the editor of the drug library, or the user of a pump.

While the embodiments of the invention disclosed herein are presently considered to be preferred, various changes and modifications can be made without departing from the scope of the invention. Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above. The scope of the invention is indicated in the appended claims, and all changes that come within the meaning and range of equivalents are intended to be embraced therein.

The following is claimed:

1. A medical intravenous infusion pump system comprising:
an electronic processor with an electronic memory;
an electrical power cable or battery;
at least first and second electromechanical pump drivers, each pump driver configured to receive at least one disposable fluid holder that is connectable to one or more fluid lines from one or more fluid source containers, and each pump driver configured to pump medical fluid through the fluid holders;
an electronic display configured to detect a user's touch selection, the display comprising first and second display regions, each of the display regions corresponding to one of the first or second pump drivers, the first display region comprising a first association graphic and the second display region comprising a second association graphic, each of the association graphics comprising a spatial or directional indicator in the form of an arrow pointing in the direction of the corresponding first or second pump driver, each arrow configured to help associate in a user's mind a correlation between the user's touch selection and the corresponding pump driver;
a first product graphic and a second product graphic displayed on the electronic display, the first and second product graphics representing the fluid holders configured to be received by the first and second pump drivers; and
a first machine-readable code displayed within the first association graphic and a second machine-readable code positioned within the second association graphic, each of the machine-readable codes comprising a QR code or a bar code, the first and second machine-readable codes configured to help coordinate information exchange between the medical intravenous infusion pump system and a different computer system by enabling encoded information to be read by an optical reader.

2. The combination of the medical intravenous infusion pump system of claim 1 and the disposable fluid holder.

3. The combination of claim 2, wherein the disposable fluid holder is a cassette, a section of tubing, or a syringe.

4. The combination of the medical intravenous infusion pump system of claim 1, and the one or more fluid lines or the one or more fluid source containers.

5. The combination of the medical intravenous infusion pump system of claim 1 and the different computer system or the optical reader.

6. The system of claim 1, further comprising third and fourth association graphics, each of the third and fourth association graphics configured to help associate in a user's mind a correlation between the user's touch selection and a corresponding input on a corresponding fluid holder in a corresponding pump driver.

7. The system of claim 6, further comprising third and fourth machine readable codes, the third machine readable code configured to be displayed within the third association graphic, and the fourth machine readable code configured to be displayed within the fourth association graphic.

8. The system of claim 1, wherein one or more of the product graphics includes one or more connection points configured to help associate the user's selection with a corresponding physical configuration of tubing or fluid communication between the corresponding fluid holder and the corresponding fluid source containers.

9. A medical intravenous infusion pump system comprising:
an electronic processor with an electronic memory;
an electrical power cable or battery;
at least first and second electromechanical pump drivers, each pump driver configured to receive at least one disposable fluid holder that is connectable to one or more fluid lines from one or more fluid source containers, and each pump driver configured to pump medical fluid through the fluid holders;
an electronic display configured to detect a user's touch selection, the display comprising first and second display regions, each of the display regions corresponding to one of the first or second pump drivers, the first display region comprising a first association graphic and the second display region comprising a second association graphic, each of the association graphics comprising a spatial or directional indicator in the form of an arrow pointing in the direction of the corresponding first or second pump driver, each arrow configured to help associate in a user's mind a correlation between the user's touch selection and the corresponding pump driver; and
a first machine-readable code displayed within the first association graphic and a second machine-readable code positioned within the second association graphic, each of the machine-readable codes comprising a QR code or a bar code, the first and second machine-readable codes configured to help coordinate information exchange between the medical intravenous infusion pump system and a different computer system by enabling encoded information to be read by an optical reader.

10. The combination of the medical intravenous infusion pump system of claim 9 and the disposable fluid holder.

11. The combination of claim 10, wherein the disposable fluid holder is a cassette, a section of tubing, or a syringe.

12. The combination of the medical intravenous infusion pump system of claim 9, and the one or more fluid lines or the one or more fluid source containers.

13. The combination of the medical intravenous infusion pump system of claim 9 and the different computer system or the optical reader.

14. The system of claim 9, further comprising third and fourth association graphics, each of the third and fourth association graphics configured to help associate in a user's mind a correlation between the user's touch selection and a corresponding input on a corresponding fluid holder in a corresponding pump driver.

15. The system of claim 14, further comprising third and fourth machine readable codes, the third machine readable code configured to be displayed within the third association graphic, and the fourth machine readable code configured to be displayed within the fourth association graphic.

16. A medical intravenous infusion pump system comprising:
an electronic processor with an electronic memory;
an electrical power cable or battery;
at least first and second electromechanical pump drivers, each pump driver configured to receive at least one disposable fluid holder that is connectable to one or more fluid lines from one or more fluid source containers, and each pump driver configured to pump medical fluid through the fluid holders;
an electronic display configured to detect a user's touch selection, the display comprising first and second display regions, each of the display regions corresponding to one of the first or second pump drivers, the first display region comprising a first association graphic and the second display region comprising a second association graphic shaped or positioned to help associate in a user's mind a correlation between the user's touch selection and the corresponding pump driver; and
a first machine-readable code associated with the first association graphic and a second machine-readable code associated with the second association graphic, the first and second machine-readable codes configured to help coordinate information exchange between the medical intravenous infusion pump system and a different computer system.

17. The combination of the medical intravenous infusion pump system of claim 16 and the disposable fluid holder.

18. The combination of claim 17, wherein the disposable fluid holder is a cassette, a section of tubing, or a syringe.

19. The combination of the medical intravenous infusion pump system of claim 16 and the one or more fluid lines, and the one or more fluid source containers.

20. The combination of the medical intravenous infusion pump system of claim 16 and the different computer system or the optical reader.

21. The system of claim 16, further comprising third and fourth association graphics, each of the third and fourth association graphics configured to help associate in a user's mind a correlation between the user's touch selection and a corresponding input on a corresponding fluid holder in a corresponding pump driver.

22. The system of claim 16, further comprising third and fourth machine readable codes, the third machine readable code configured to be associated with the third association graphic, and the fourth machine readable code configured to be associated with the fourth association graphic.
